(19) United States
(12) Patent Application Publication
(10) Pub. No.: US 2009/0093015 A1
(43) Pub. Date: Apr. 9, 2009

(54) Beta-cryptoxanthin production using a novel lycopene beta-monocyclase gene

(75) Inventors: Michael Tai-Man Louie, Iowa City, IA (US); Emily-Jean Fuerst, Ames, IA (US)

Correspondence Address: DAVIS, BROWN, KOEHN, SHORS & ROBERTS, P.C.;THE DAVIS BROWN TOWER, 215 10TH STREET SUITE 1300, DES MOINES, IA, 50309, US

(73) Assignee: Kemin Foods, L.C., Des Moines, IA (US)

(21) Appl. No.: 11/973,546

(22) Filed: Oct. 9, 2007

(51) Int. Cl. C12P 23/00; C12N 15/11; C12N 5/04; C12P 21/04

(52) U.S. Cl. 435/67; 536/23.1; 435/410; 435/69.1; 536/23.2

(57) ABSTRACT

Novel lycopene beta-monocyclase genes were identified and used to transform a host cell to produce β-cryptoxanthin. The host cell produces lycopene and is transformed to express the novel lycopene β-monocyclase that converts lycopene into γ-carotene. The host cell is further transformed to express a lycopene hydroxylase that hydroxylates γ-carotene to 3-hydroxy-γ-carotene and a lycopene β-bicyclase that converts 3-hydroxy-γ-carotene to β-cryptoxanthin. The host cell is grown under conditions whereby γ-carotene is produced which is hydroxylated to 3-hydroxy-γ-carotene and which is converted into β-cryptoxanthin.

### BACKGROUND OF THE INVENTION

The invention relates generally to the production of carotenoids and, more specifically, to the use of novel lycopene β-monocyclase genes to produce β-cryptoxanthin and other asymmetric carotenoids.

Carotenoids are naturally occurring pigments synthesized by plants, bacteria, and fungi. These pigments have protective functions against oxidative damage by quenching harmful singlet oxygen, reactive oxygen species and free radicals [Krinsky N. I. (1994) The biological properties of carotenoids. Pure Appl. Chem. 66:1003-1010] that are metabolic by-products in cells. Lutein, zeaxanthin, and β-cryptoxanthin (BCX) are members of the carotenoid family referred to as xanthophyll. Xanthophylls possess one or more oxygenated groups (hydroxyl- or keto-group), separating them from other non-oxygenated acyclic and cyclic carotenoids such as lycopene and β-carotene. β-cryptoxanthin is a unique xanthophyll, which differs from lutein and zeaxanthin in that it is an asymmetrical, monohydroxylated xanthophyll (FIG. 1).

β-cryptoxanthin is one of the most abundant carotenoids found in human serum [Khachik F., Spangler C. J., and Smith J. C. (1997) Identification, quantification, and relative concentrations of carotenoids and their metabolites in human milk and serum. Anal. Chem. 69:1873-1881] and it also can be found in various human tissues [Khachik et al. (1997); Yeum, K. J., Ahn S. H., Rupp de Pavia S. A., Lee-Kim Y. C., Krinsky N. I., and Rusell R. M. (1998) Correlation between carotenoid concentrations in serum and normal breast adipose tissue of women with benign breast tumor or breast cancer. J. Nutr. 128:1920-1926; Wingerath T., Sies, H., and Stahl W. (1998) Xanophyll esters in human skin. Arch. Biochem. Biophys. 355:271-274]. Natural sources of β-cryptoxanthin include oranges, tangerines, papayas, peaches, mangoes, and red sweet peppers. Numerous scientific studies support the idea that BCX is beneficial to human health. High pre-diagnostic levels of β-cryptoxanthin in serum have been found to be associated with reduced risk of lung cancer in a recent cohort study [Yuan J. M., Ross R. K., Chu X., Gao Y. T., and Yu M. C. (2001) Prediagnostic levels of serum β-cryptoxanthin and retinal predict smoking-related lung cancer risk in Shanghai, China. Cancer Epidemiol. Biomark. Prev. 10:767-773; Yuan J. M., Stram D. O., Arakawa K., Lee H. P., and Yu M. C. (2003) Dietary cryptoxanthin and reduced risk of lung cancer: the Singapore Chinese Health Study. Cancer Epidemiol. Biomark. Prev. 12:890-898]. Furthermore, experimental and epidemiological data support the utility of dietary β-cryptoxanthin as a preventive agent against the risk of developing prostate cancer, colon cancer [Bhosale P. and Bernstein P. S. (2005) Microbial xanophylls. Appl. Microbiol. Biotechnol. 68:445-455], and rheumatoid arthritis [Pattison D. J., Symmons D. P. M., Lunt M., Welch, A., Bingham S. A., Day N. E., and Silman A. J. (2005) Dietary β-cryptoxanthin and inflammatory polyarthritis: results from a population-based prospective study. Am. J. Clin. Nutr. 82:451-455]. BCX has been shown to stimulate osteoblastic bone formation and to inhibit osteoclastic bone resorption in vitro [Yamaguchi M. and Uchiyama S. (2004) β-Cryptoxanthin stimulates bone formation and inhibits bone resorption in tissue culture in vitro. Mol. Cell. Biochem. 258:137-144; Uchiyama S, and Yamaguchi M. (2005) β-Cryptoxanthin stimulates cell proliferation and transcriptional activity in osteoblastic MC3T3-E1 cells. Int. J. Mol. Med. 15:675-681; Uchiyama S, and Yamaguchi M. (2005) β-cryptoxanthin stimulates cell differentiation and mineralization in osteoblastic MC3T3-E1 cells. J. Cell. Biochem. 95:1224-1234; Uchiyama S. and Yamaguchi M. (2004) Inhibitory effect of beta-cryptoxanthin on osteoclast-like cell formation in mouse marrow cultures. Biochem. Pharmacol. 67:1297-1305;]. Animal studies demonstrated that oral administration of β-cryptoxanthin was associated with the prevention of bone loss due to increasing age and osteoporosis [Uchiyama S., Sumida T., and Yamaguchi M. (2004) Oral administration of β-cryptoxanthin induces anabolic effects on bone components in the femoral tissues of rats in vivo. Biol. Pharm. Bull. 27:232-235; Uchiyama S., Sumida T., and Yamaguchi M. (2004) Anabolic Effect of β-Cryptoxanthin on Bone Components in the Femoral Tissues of Aged Rats in vivo and in vitro. J. Health Sci. 50:491-496; Uchiyama S. and Yamaguchi M. (2005) Oral administration of β-cryptoxanthin prevents bone loss in streptozotocin-diabetic rats in vivo. Biol. Pharm. Bull. 28:1766-1769]. Despite these potential health benefits, β-cryptoxanthin is currently unavailable as a dietary supplement ingredient partly because its concentration found in natural sources is extremely low, with a highest reported concentration of about 0.005% (w/w) [Kim I. J., Ko K. C., Ko C. S., and Chung W. I. (2001) Isolation and characterization of cDNAs encoding β-carotene hydroxylase in Citrus. Plant Sci. 161:1005-1010]. Therefore, production of β-cryptoxanthin by solvent-based extraction procedures from natural plant sources is not commercially viable. A recent study reported that carotenoids extracted from Flavobacterium lutescens ITCB008 biomass were 95% β-cryptoxanthin, with a yield of 770 mg β-cryptoxanthin per kg dry biomass [Serrato-Joya O., Jimenez-Islas H., Botello-Alverez E., Rico-Martinez R., and Navarrete-Bolanos J. L. (2006) Production of β-cryptoxanthin, a provitamin-A precursor, by Flavobacterium lutescens in J. Food Sci. 71:E314-E319]. However, this production level is not sufficient for commercial production of β-cryptoxanthin. Currently, no other microorganisms are known to accumulate a high level of β-cryptoxanthin naturally. The lack of a microbial source that naturally produces a high level of β-cryptoxanthin as the final product also prohibits using fermentation technology for commercial production of this molecule. Recently, Khachik patented a chemical synthetic method for production of BCX from lutein [U.S. Pat. No. 6,911,564]. This technology has been utilized at a pilot scale facility, yielding a product consisting of approximately 20% β-cryptoxanthin [Liu Y., Zhang F., Showalter H. A., Scroggins R., Stomp R., Miller H., and DeFreitas Z. (2005) Campaign report for the third pilot GMP production trial for the preparation of β-cryptoxanthin. (Unpublished)]. Purification technology was also developed to enrich β-cryptoxanthin from the crude product, with the final product containing 45% β-cryptoxanthin and over 90% carotenoids (U.S. Patent Application Ser. No. 60/921,913, filed Apr. 5, 2007).

The present invention includes in preferred embodiments the successful production of β-cryptoxanthin through this engineered pathway in recombinant E. coli cells.

### SUMMARY OF THE INVENTION

This invention describes the use of lycopene β-monocyclase ultimately for the production of β-cryptoxanthin. The cloned lycopene β-monocyclase genes are significantly different from known lycopene β-monocyclase genes and the application of a lycopene β-monocyclase in a β-cryptoxanthin biosynthetic pathway is not known in the art. Host cells of bacteria, yeasts, filamentous fungi, algae, and green plants that produce lycopene are transformed to express the lycopene β-monocyclase and grown under conditions wherein the lycopene β-monocyclase first converts lycopene to γ-carotene. A CrtZ (β-carotene hydroxylase) then hydroxylates the single β-ionone ring of γ-carotene to form 3-hydroxyl γ-carotene. Finally, an inducible lycopene β-cyclase (CrtY) is used to cyclize the linear end of 3-hydroxyl γ-carotene to form β-cryptoxanthin. Preferred bacteria include Escherichia. Preferred green plants include corn, soybeans, alfalfa, Arabidopsis, sorghum, wheat, barley, oats, carrot, pumpkin, pepper and rice.

### BRIEF DESCRIPTION OF THE DRAWINGS

FIG. 1 is a drawing of chemical structures of some common xanthophylls and non-oxygenated carotenoids.

FIG. 2 is a drawing of an engineered metabolic pathway of the conversion of lycopene to BCX.

FIGS. 3A-F are HPLC chromatograms of cell extracts from lycopene-producing E. coli JM109 (pACmod-EBI₁₄) with (A) no plasmid, (B) pUCmod-766-8, (C) pUCmod-766-F2-6, and (D) pUCmod-766-F3-2, after the cultures were grown for 24 h; HPLC chromatograms of (E) γ-carotene and (F) β-carotene are also shown.

FIGS. 4A-D are absorption spectra of various HPLC peaks with absorption maxima labeled (in nm); (A) Peak 1 in FIGS. 3C and D; (B) γ-carotene; (C) first half of Peak 2 in FIGS. 3C and 3D; and (D) second half of Peak 2 in FIGS. 3C and 3D.

FIGS. 5A-B are HPLC chromatograms of cell extracts from lycopene-producing E. coli JM109 (pACmod-EBI₁₄) with: (A) pUCmod-DgcrtLm-F1-G and (B) pUCmod-DgcrtLm-F2-A, after cultures were grown for 24 h; peak A is γ-carotene; peak B is possibly torulene and the identity of Peak C is currently unknown.

FIG. 6 is a drawing of a metabolic pathway that explains the production of torulene.

FIG. 7 is an HPLC analysis of cell extracts from lycopene-producing E. coli JM109 (pACmod-EBI) with (A) no plasmid, (B) pUCmod-DgcrtLm-F2-A, (C) pUCmod-766-F2-6, after the cultures were grown for 24 h.

FIG. 8 is an HPLC analysis of cell extracts from E. coli JM109 (pACmod-EBIZ) with (A) pUCmod-DgcrtLm-F2-A and (B) pUCmod-766-F2-6, after the cultures were grown for 24 h

FIG. 9 is an HPLC analysis of cell extracts from E. coli JM109 (pACmod-EBIZ/pUCmod-766-F2-6/pBBR1-crtY) (A) before and (B) 6 hours after induction by L-arabinose.

FIG. 10 is a multiple sequence alignment of lycopene β-cyclases; only part of the alignment is shown with the five conserved domains found in classical CrtY-type lycopene cyclase are labeled A to E; amino acid identities are highlighted in black and conserved amino acids in gray; the corresponding sequences are: Rhodococcus sp. RHA1 ORF766 (RHA-766), R. erythropolis CrtLm (Re-CrtLm), D. geothermalis ORF2206 (Dg-ORF2206), D. radiodurans CrtLm (Dr-CrtLm), Synechococcus elongates CrtL (Se-CrtL), Synechococcus sp. CrtL (Syne-sp-CrtL), Arabidopsis thaliana Lyc-B (At-LycB), Pantoea ananatis CrtY (Pa-CrtY), and Agrobacterium aurantiacum CrtY (Agau-CrtY).

### DETAILED DESCRIPTION OF PREFERRED EMBODIMENTS

Carotenoids are a diverse group of natural pigments produced by plants, bacteria, yeasts and fungi. These pigments play an important protective function in quenching harmful singlet oxygen molecules, reactive oxygen species, and free radicals that are metabolic by-products in cells that cause oxidative damage. Hydroxylated carotenoids, such as lutein, zeaxanthin, and β-cryptoxanthin, are members of the xanthophyll class of carotenoids. β-cryptoxanthin differs from its xanthophyll counterparts by having only one rather than two hydroxyl groups, and hence it is an asymmetric molecule.

There is interest in the use of β-cryptoxanthin as a dietary supplement ingredient because of its healthful benefits. However, the low available concentration of β-cryptoxanthin in natural sources prevents commercialization of this molecule by traditional solvent-based extraction procedures. Natural sources of β-cryptoxanthin include fruits such as oranges, tangerines, papayas, and mangos. β-cryptoxanthin is produced in trace amounts in these natural sources, with the highest reported concentration of approximately 0.005% (w/w). A recent study reported that carotenoids extracted from Flavobacterium lutescens ITCB008 biomass were 95% β-cryptoxanthin, with a yield of 770 mg β-cryptoxanthin per kg dry biomass [Serrato-Joya et al., 2006]. However, this production level is not sufficient for commercial production of β-cryptoxanthin. Currently, no other microorganisms are known to accumulate a high level of β-cryptoxanthin naturally. The lack of a microorganism that naturally produces a high level of beta-cryptoxanthin as the final product also prohibits using fermentation technology for commercial production of β-cryptoxanthin.

The reason why β-cryptoxanthin is rarely accumulated to a high level in any natural sources lies in the natural biosynthetic pathway for xanthophylls. All xanthophylls are synthesized from basic 5-carbon isoprenoid compounds, isopentenyl pyrophosphate (IPP) and dimethylallyl pyrophosphate (DMAPP). One IPP molecule and one DMAPP molecule condense to form geranyl diphosphate (GPP), which is elongated to farnesyl diphosphate (FPP), geranylgeranyl diphosphate (GGPP), and finally the 40-carbon phytoene. In bacteria and fungi, a single phytoene desaturase (CrtI) catalyzes sequential desaturation of phytoene to lycopene. In many organisms, desaturation is followed by cyclization. Cyclization of lycopene by a β-cyclase (CrtY) leads to the formation of the bicyclic carotenoid beta-carotene. β-carotene can then be either hydroxylated by a β-carotene hydroxylase (CrtZ) or oxidized by a β-carotene ketolase. CrtZ is an enzyme that sequentially hydroxylates both β-ionone rings of β-carotene to zeaxanthin, with β-cryptoxanthin as an intermediate. Because of this property of CrtZ, the intermediate β-cryptoxanthin molecule rarely accumulates to high levels in any natural sources.

Most known lycopene β-cyclases (CrtY) are bicyclases because they catalyze the sequential formation of β-ionone rings at both ends of lycopene. Recently, three lycopene β-monocyclase genes were identified in marine bacterium strain P99-3, and in two non-photosynthetic bacteria, Rhodococcus erythropolis AN12 and Deinococcus radiodurans R1. These three gene products, when independently expressed as recombinant proteins in E. coli, cyclized only one end of lycopene and produced the monocyclic γ-carotene. It was conceived that a lycopene β-monocyclase could be applied to an engineered biosynthetic pathway for β-cryptoxanthin. In this pathway, a lycopene β-monocyclase first converts lycopene to γ-carotene. A common CrtZ (β-carotene hydroxylase) then hydroxylates the single β-ionone ring of γ-carotene and results in 3-hydroxyl γ-carotene. Finally, an inducible lycopene β-cyclase (CrtY) is used to cyclize the linear end of 3-hydroxyl γ-carotene to form β-cryptoxanthin. This last step is carried out under anoxic conditions that prevent hydroxylation of the newly formed β-ionone ring by existing CrtZ activity. The advantage of this proposed pathway is that β-cryptoxanthin would be the final product and would not be further metabolized to zeaxanthin.

Scientists at E. I. DuPont de Nemours Inc. identified the two lycopene β-monocyclase genes in Rhodococcus erythropolis AN12 and Deinococcus radiodurans R1 (U.S. Pat. No. 7,063,955). Scientists at the Marine Biotechnology Institute of Japan discovered the lycopene β-monocyclase gene in marine bacterium strain P99-3 (Pat. App. No. JP2004154061). The present invention relates to new lycopene β-monocyclase genes used in the above-mentioned engineered pathway for β-cryptoxanthin biosynthesis.

The lycopene β-monocyclase genes, crtLm, of Rhodococcus erythropolis AN12 (Genbank accession no. AY437860) and Deinococcus radiodurans R1 (Genbank accession no. AAF10377.1) were used as queries to search current microbial genome sequencing project databases by TBLASTX, which compares the six-frame translations of a nucleotide query sequence against the six-frame translations of a nucleotide sequence database. R. erythropolis crtLm showed highest homology to ORF766 in Rhodococcus sp. RHA1 genome which encodes a putative lycopene cyclase (SEQ ID NO. 1). D. radiodurans crtLm showed highest homology to ORF2206 in Deinococcus geothermalis DSM11300 genome which also encodes a putative lycopene cyclase (SEQ ID NO. 2). The deduced protein sequence of ORF766 and ORF2206 showed 52% and 61% identity, respectively, to R. erythropolis and D. radiodurans CrtLm protein sequences when ClustalW (European Bioinformatics Institute 2006, web site: ebi.ac.uk/clustalw/) were used to align the sequences. These two ORFs did not show significant homology (6-12% identity) to the lycopene β-monocyclase CrtYm identified in the marine bacterium strain P99-3. Alignment of the lycopene β-monocyclase protein sequence of Rhodococcus sp. RHA1 and R. erythropolis by EMBOSS::water, an alignment program that uses the Smith-Waterman algorithm, the two protein sequences were 53.8% identical. The lycopene β-monocyclase protein sequence of Deinococcus geothermalis DSM11300 was 60.9% identical to that of D. radiodurans when the two protein sequences were aligned by EMBOSS::water.

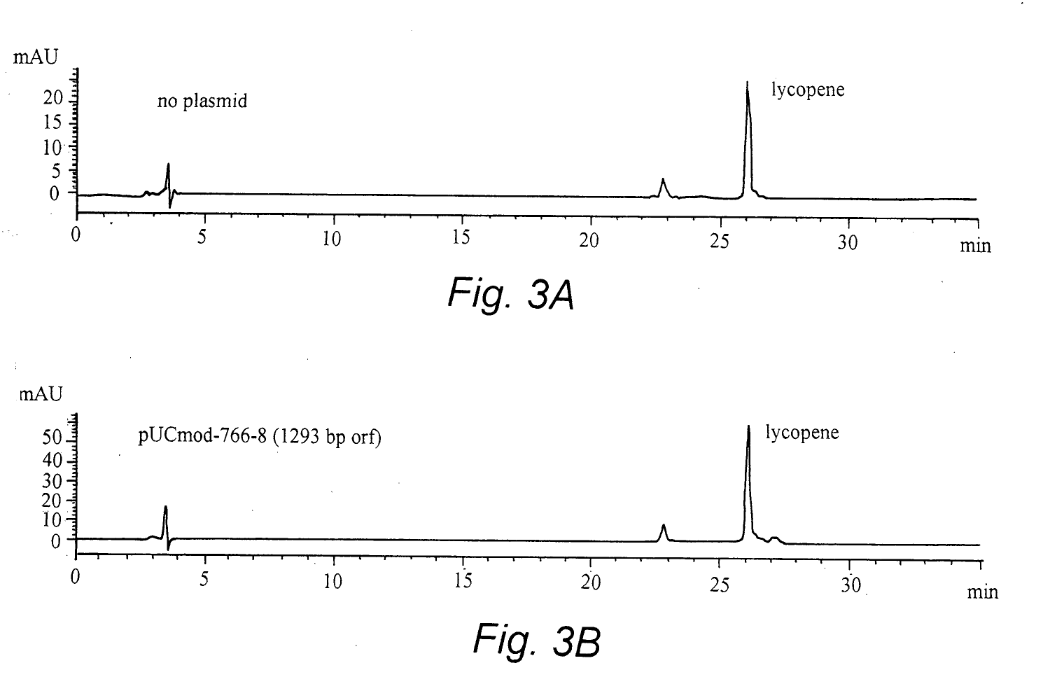

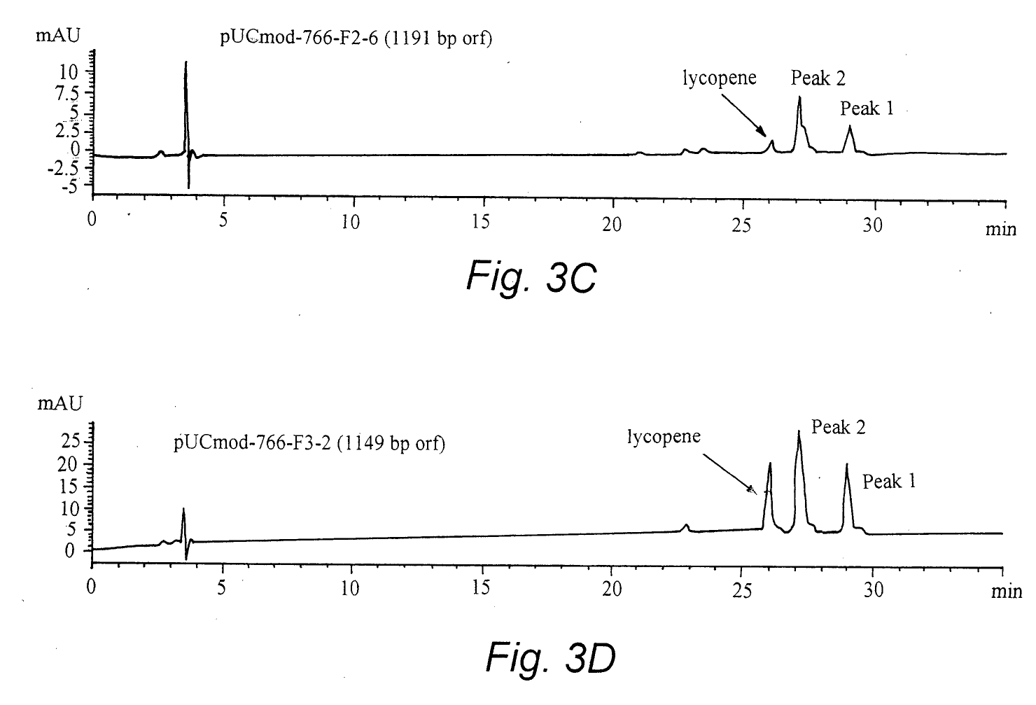

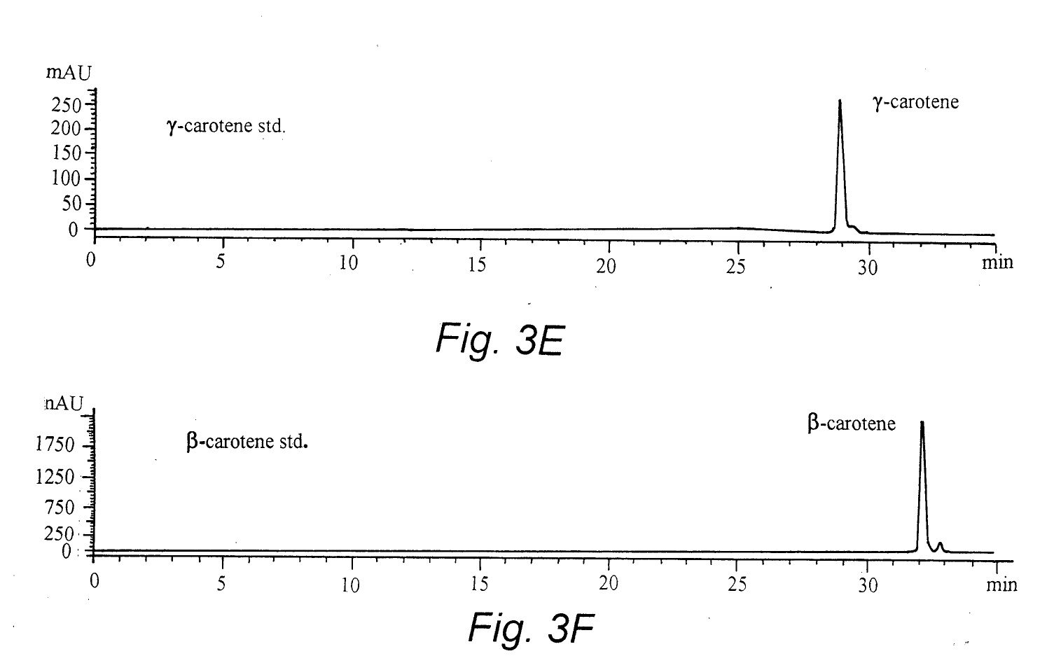

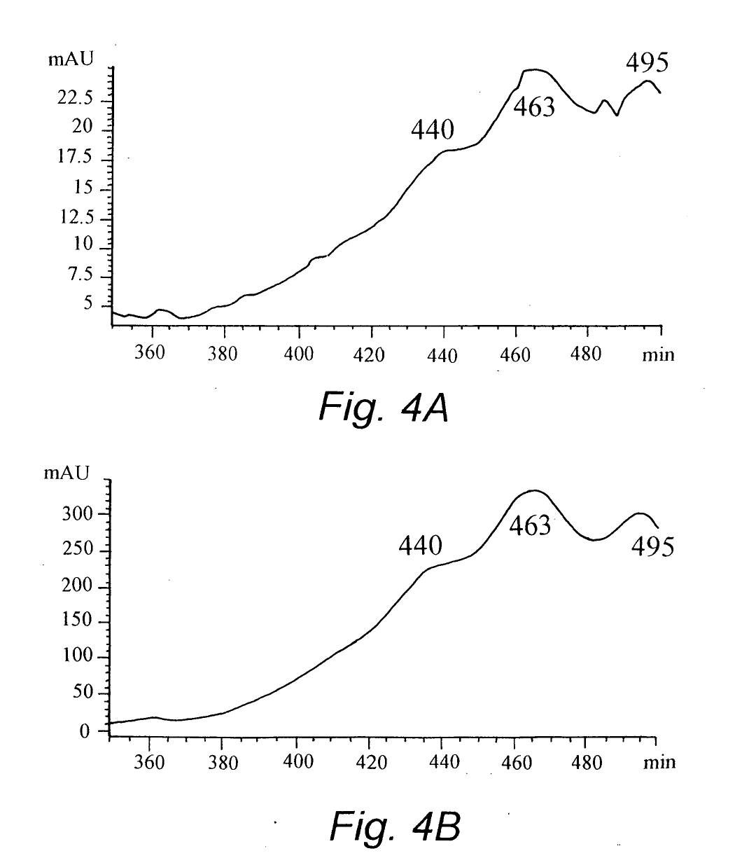

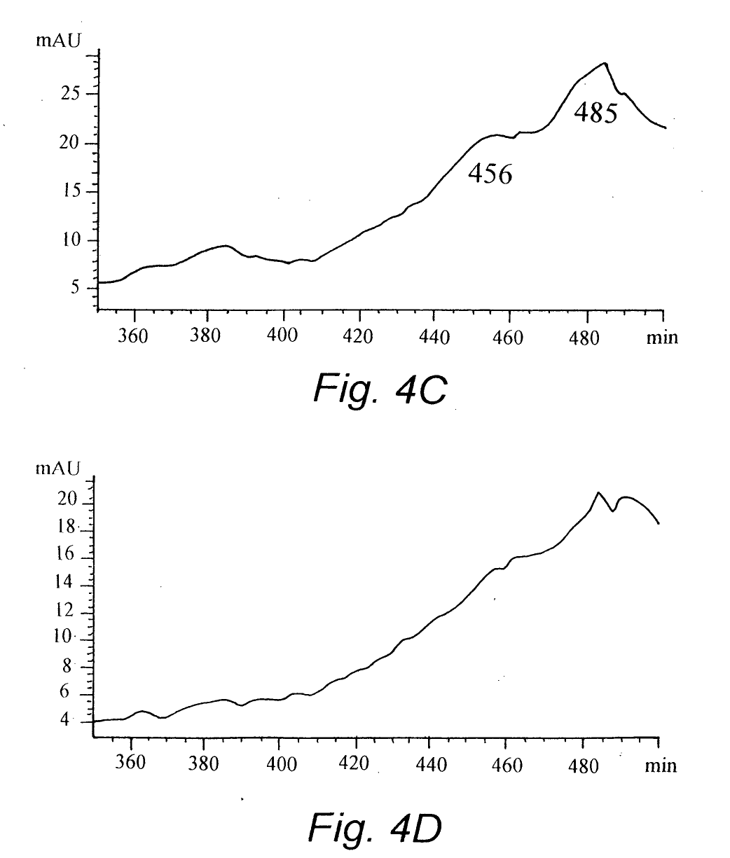

HPLC analysis of JM109 (pACmod-EBI14/pUCmod-766-F2-6) and JM109 (pACmod-EBI14/pUCmod-766-F3-2) cell extracts identified three major metabolites (FIGS. 3 and 4). One of the peaks was lycopene (retention time at about 26 min). The retention time (at about 29 min) of the second metabolite was identical to that of an authentic γ-carotene standard. Additionally, the absorption maxima of Peak 1 (440, 463, and 495 nm) were also identical to those of γ-carotene.

The main advantage of this invention is that the protein sequences of these two new lycopene β-monocyclases displayed significant differences from the two homologs described in the DuPont patent. The lycopene β-monocyclase protein sequence of Rhodococcus sp. RHA1 was 53.8% identical to that of R. erythropolis when the two protein sequences were aligned by EMBOSS::water, an alignment program that uses the Smith-Waterman algorithm. The deduced protein sequence of ORF766 showed 52% to R. erythropolis CrtLm protein sequences when ClustalW was used for sequence alignment. The lycopene β-monocyclase protein sequence of Deinococcus geothermalis DSM11300 was 60.9% identical to that of D. radiodurans when the two protein sequences were aligned by EMBOSS::water and 61% identity to D. radiodurans CrtLm protein sequences when aligned by ClustalW.

The application of a lycopene β-monocyclase in a β-cryptoxanthin biosynthetic pathway has not been previously accomplished or proposed.

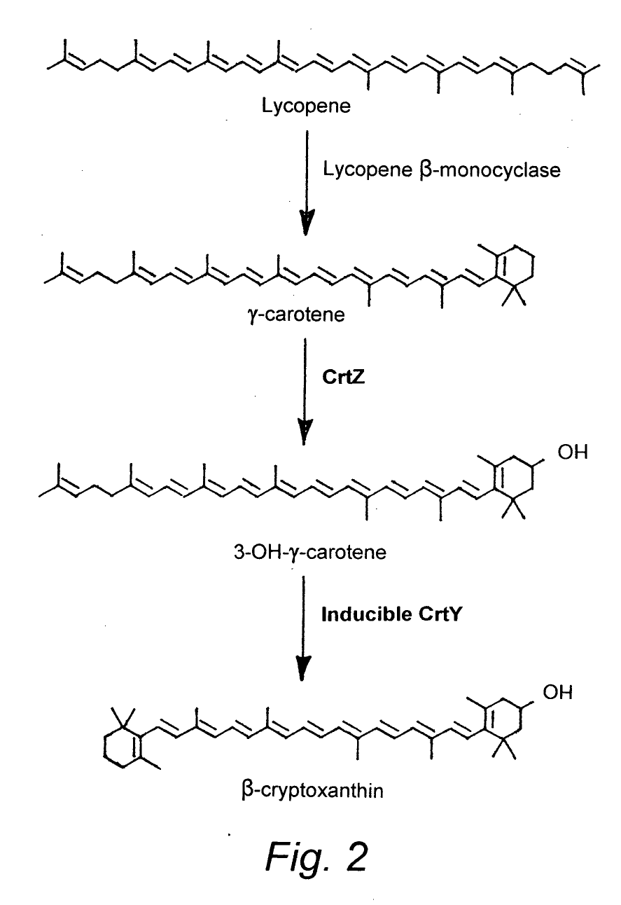

An Arabidopsis thaliana β-carotene hydroxylase gene (crtZ) was successfully cloned into an E. coli strain, which was engineered to produce β-carotene, and resulted in transformation of β-carotene to mostly β-cryptoxanthin (U.S. patent application Ser. No. 11/546,702, filed Oct. 12, 2006, and incorporated herein by this reference). In this application there is described the isolation of two lycopene β-monocyclase genes (crtLm) from Rhodococcus sp. RHA1 and Deinococcus geothermalis DSM11300. The use of this type of gene is described in an engineered metabolic pathway for β-cryptoxanthin synthesis (FIG. 2). In this pathway, a lycopene β-monocyclase converts lycopene to γ-carotene. When expressed in a microbial host, the CrtLm enzymes were shown to have the desired monocyclization activities on lycopene and produced γ-carotene. A common bacterial β-carotene hydroxylase (CrtZ) hydroxylates the single β-ionone ring of γ-carotene and results in the formation of 3-OH-γ-carotene. Finally, an inducible lycopene 13-bicyclase (CrtY) is used to cyclize the linear end (Ψ-end) of 3-OH-γ-carotene to form β-cryptoxanthin. Suitable microbial host cells include carotenoid or isoprenoid producing bacteria and carotenoid or isoprenoid producing fungi and, more specifically, include but are not limited to, Acinetobacter, Agrobacterium, Alcaligenes, Anabaena, Aspergillus, Bacillus, Brevibacterium, Candida, Chlorobium, Chromatium, Corynecbacteria, Cytophaga, Deinococcus, Erwinia, Erythrobacter, Eshcerichia, Flavobacterium, Hansenula, Klebsiella, Methanobacterium, Methylobacter, Methyloccocus, Methylocystis, Methylomicrobium, Methylomonas, Methylsinus, Mycobacterium, Myxococcus, Pantoea, Pichia, Pseudomonas, Rhodobacter, Rhodococcus, Saccharomyces, Salmonella, Sphingomonas, Streptomyces, Synechococcus, Synechocystis, Thiobacillus, Trichoderma, and Zymomonas.

Definitions for a number of the terms are used in this specification are provided.

The term “carotenoid” includes both carotenes and xanthophylls. A “carotene” refers to a hydrocarbon carotenoid. Carotene derivatives that contain one or more oxygen atoms, in the form of hydroxy-, methoxy-, oxo-, epoxy-, carboxy-, or aldehydic functional groups, or within glycosides, glycoside esters, or sulfates, are collectively known as “xanthophylls”. Carotenoids are furthermore described as being acyclic, monocyclic, or bicyclic depending on whether the ends of the hydrocarbon backbones have been cyclized to yield aliphatic or cyclic ring structures.

Carotenoid biosynthesis starts with the isoprenoid pathway to generate isopentenyl pyrophosphate (IPP). IPP was condensed with its isomer dimethylallyl pyrophophate (DMAPP) to C₁₀ geranyl pyrophosphate (GPP) and elongated to C₁₅ farnesyl pyrophosphate (FPP). FPP synthesis is common in both carotenogenic and non-carotenogenic bacteria. Subsequent enzymes in the carotenoid pathway generate carotenoid pigments from the FPP precursor and can be divided into two categories: carotene backbone synthesis enzymes and subsequent modification enzymes. The backbone synthesis enzymes include geranylgeranyl pyrophosphate synthase (CrtE), phytoene synthase (CrtB), phytoene dehydrogenase (CrtI) and lycopene cyclase (CrtY/L), etc. The modification enzymes include ketolases, hydroxylases, dehydratases, glycosylases, etc.

Two types of lycopene cyclases (β-cyclases and ε-cyclases) have been reported (Cunningham et al., Plant Cell. 8:1613 1626 (1996)). All previously described lycopene β-cyclases catalyze the formation of β-ionone rings from Ψ end groups found on acyclic carotenoids such as lycopene (Ψ, Ψ-carotene), usually resulting in a symmetrical bicyclic product such as β-carotene. The lycopene ε-cyclases, usually found in plants, catalyze the formation of ε-ionone rings from the Ψ end groups. Most lycopene ε-cyclases catalyze formation of the asymmetric monocyclic δ-carotene (Ψ, ε-carotene). A lycopene ε-cyclase from lettuce catalyzes the formation of bicyclic ε-carotene (ε,ε-carotene) (Cunningham et al., PNAS, 98:2905 2910, (2000)). The difference between the β-ionone and ε-ionone ring structure is based on the location of the double bond within the 6-member ring.

The terms “lycopene β-monocyclase” or “β-monocyclase” will be used interchangeably and refer to an enzyme that catalyzes the formation of a β-ionone ring cyclic end group from the acyclic Ψ end group.

The term “CrtE” refers to geranylgeranyl pyrophosphate synthase enzyme encoded by crtE gene which converts trans-trans-farnesyl diphosphate+isopentenyl diphosphate to pyrophosphate+geranylgeranyl diphosphate.

The term “CrtB” refers to phytoene synthase enzyme encoded by crtB gene that catalyzes reaction from prephytoene diphosphate (geranylgeranyl pyrophosphate) to phytoene.

The term “CrtI” refers to phytoene dehydrogenase enzyme encoded by crtI gene that converts phytoene into lycopene via the intermediaries of phytofluene, zeta-carotene and neurosporene by the introduction of 4 double bonds.

The term “CrtY” refers to lycopene cyclase enzyme encoded by crtY gene that converts lycopene to beta-carotene.

The term “CrtZ” refers to the β-carotene hydroxylase enzyme encoded by crtZ gene which catalyses hydroxylation reaction from β-carotene to zeaxanthin.

The term “CrtLm” refers to lycopene β-monocyclase enzyme encoded by crtLm gene that catalyses the formation of a single β-ionone ring from a Ψ end group found on acyclic carotenoids such as lycopene (Ψ, Ψ-carotene), resulting in an asymmetrical monocyclic product such as γ-carotene.

The term “CrtI₁₄” refers to a mutated phytoene dehydrogenase enzyme encoded by crtI₁₄ gene that converts phytoene into 3,4,3′,4′-tetradehydrolycopene via the intermediaries of phytofluene, zeta-carotene, neurosporene, lycopene, and 3,4-didehydrolycopene by the introduction of 6 double bonds.

“Dg-crtLm-F1” represents nucleic acids encoding a partial sequence of crtLm from Deinococcus geothermalis used in the method of the invention for amplification of the crtLm gene.

“Dg-crtLm-F2” ” represents nucleic acids encoding a partial sequence of crtLm from Deinococcus geothermalis used in the method of the invention for amplification of the crtLm gene.

“Dg-crtLm-R2” ” represents nucleic acids encoding a partial sequence of crtLm from Deinococcus geothermalis used in the method of the invention for amplification of the crtLm gene.

“Rha766-F” ” represents nucleic acids encoding a partial sequence of crtLm from Rhodococcus RHA1 used in the method of the invention for amplification of the crtLm gene.

“Rha766-R” represents nucleic acids encoding a partial sequence of crtLm from Rhodococcus RHA1 used in the method of the invention for amplification of the crtLm gene.

“Rha-1 mut” represents nucleic acids encoding a partial sequence of Rhodococcus RHA1 crtLm from pUCmod-Rha766 used in the method of the invention for mutation correction and amplification of the crtLm gene.

“Rha-2 mut” represents nucleic acids encoding a partial sequence of Rhodococcus RHA1 crtLm from pUCmod-Rha766 used in the method of the invention for mutation correction and amplification of the crtLm gene.

“New766-F” represents nucleic acids encoding a partial sequence of Rhodococcus RHA1 crtLm from pUCmod-766-SDM4 used in the method of the invention for amplification of the crtLm gene.

“New766-R” represents nucleic acids encoding a partial sequence of Rhodococcus RHA1 crtLm from pUCmod-766-SDM4 used in the method of the invention for amplification of the crtLm gene.

“New766-F2” represents nucleic acids encoding a partial sequence of Rhodococcus RHA1 crtLm from pUCmod-766-8 used in the method of the invention for amplification of the crtLm gene.

“New766-F3” represents nucleic acids encoding a partial sequence of Rhodococcus RHA1 crtLm from pUCmod-766-8 used in the method of the invention for amplification of the crtLm gene.

“crtI-F” represents nucleic acids encoding a partial sequence of crtI from Pantoea ananatis used in the method of the invention for amplification of the crtI gene.

“crtI-R” represents nucleic acids encoding a partial sequence of crtI from Pantoea ananatis used in the method of the invention for amplification of the crtI gene.

“crtI-mutF” represents nucleic acids encoding a partial sequence of crtI from Pantoea ananatis used in the method of the invention for mutation correction and amplification of the crtI gene.

“crtI-mutR” represents nucleic acids encoding a partial sequence of crtI from Pantoea ananatis used in the method of the invention for mutation correction and amplification of the crtI gene.

“Plac-crtI-F” represents nucleic acids encoding a partial sequence of the modified-lac promoter from the vector pUCmod used in the method of the invention for amplification of the Plac-crtI gene cassette.

“Plac-crtI-R” represents nucleic acids encoding a partial sequence of crtI from the vector pUCmod used in the method of the invention for amplification of the Plac-crtI gene cassette.

“Plac-766-F” ” represents nucleic acids encoding a partial sequence of the modified-lac promoter from the vector pUCmod-Pa-crtZ used in the method of the invention for amplification of the Plac-crtZ gene cassette.

“Plac-766-R” represents nucleic acids encoding a partial sequence of Pantoea ananatis crtZ from the vector pUCmod-Pa-crtZ used in the method of the invention for amplification of the Plac-crtZ gene cassette.

“pBAD-Y-F” represents nucleic acids encoding a partial sequence of Pantoea ananatis crtY from the vector pUCmod-crtY used in the method of the invention for amplification of the crtY gene.

“pBAD-Y—R” represents nucleic acids encoding a partial sequence of Pantoea ananatis crtY from the vector pUCmod-crtY used in the method of the invention for amplification of the crtY gene.

“pUCmod-F” represents nucleic acids encoding a partial sequence of the pUCmod vector used in the method of the invention for amplification of inserted nucleic acids.

“pUCmod-R” ” represents nucleic acids encoding a partial sequence of the pUCmod vector used in the method of the invention for amplification of inserted nucleic acids

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. For example a common set of stringent conditions consists of hybridization at 0.1×SSC, 0.1% SDS, 65° C. and washed with 2×SSC, 0.1% SDS followed by 0.1×SSC, 0.1% SDS. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. Hybridization requires that the two nucleic acids contain complementary sequences although, depending on the stringency of the hybridization, mismatches between bases are possible. The appropriate stringency for hybridizing nucleic acids depends on the length of the nucleic acids and the degree of complementation, variables well known in the art. The relative stability (corresponding to higher Tm) of nucleic acid hybridization decreases in the following order: RNA:RNA, DNA:RNA, DNA:DNA. For hybrids of greater than 100 nucleotides in length, equations for calculating Tm have been derived (see Sambrook et al., supra, 9.50 9.51). For hybridizations with shorter nucleic acids, i.e., oligonucleotides, the position of mismatches becomes more important, and the length of the oligonucleotide determines its specificity (see Sambrook et al., supra, 11.7 11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times, are commonly referred to as “constitutive promoters”. It is further recognized that since in most cases the exact boundaries of regulatory sequences have not been completely defined, DNA fragments of different lengths may have identical promoter activity.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The terms “plasmid” and “vector” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “expression cassette” is used in the present application to refer to a foreign gene having elements in addition to foreign gene(s) that allow for enhanced expression of that gene in a foreign host.

The term “ligation” refers to a process by which two strands of DNA are joined.

A “broad-host-range” plasmid refers to the ability of the plasmid to enter and express genes into functional proteins in multiple host organisms, usually as a result of an operon that is present within the plasmid sequence.

The term “digest” means restriction digest, that is, the process of cutting DNA molecules with special enzymes called restriction endonucleases.

A “β-ionone ring” is a six carbon ring structure, in which a double bond is found between carbon 5 and 6, and is formed by a β-cyclase that converts Ψ end groups found on acyclic carotenoids such as lycopene (Ψ, Ψ-carotene).

An “inducible” promoter is not always active and requires physical or chemical activation. IPTG is a classic example of a compound added to cells to activate a promoter. It is often used to activate the lacZ gene when cloning a fragment of DNA and using blue/white selection. IPTG can be added to the cells to activate the downstream gene or removed to inactivate the gene.

An “operon” is a group of key nucleotide sequences including an operator, a promoter, and one or more structural genes that are controlled as a unit to produce messenger RNA (mRNA).

### Example 1

Materials. All reagents were of the highest purity available and were purchased from Sigma (St. Louis, Mo.) Aldrich (Milwaukee, Wis.), and Fisher Scientific (Pittsburgh, Pa.) unless otherwise noted. PCR primers were purchased from Integrated DNA Technologies (Coralville, Iowa). Pfu DNA polymerase (Stratagene, La Jolla, Calif.), Taq DNA polymerase (Fisher), and FailSafe™ PCR enzyme mix (Epicentre, Madison, Wis.) were used in PCR reactions. Restriction endonucleases were purchased from Invitrogen (Carlsbad, Calif.), New England Biolabs (Beverly, Mass.), and Fermentas (Hanover, Mass.). Fast-Link™ DNA ligation kit was purchased from Epicentre. γ-carotene was purchased from Carotenature (Lupsingen, Switzerland).

Bacterial strains and plasmids. The bacterial strains and plasmids used in this study were listed in Table 1.

Genomic DNA preparations. Deinococcus geothermalis DSM 11300 was grown in Degryse medium 162 [Ferreira A. C., Nobre M. F., Rainey F. A., Silva M. T., Wait R., Burghardt J., Chung A. P., and da Costa M. S. (1997) Deinococcus geothermalis sp. nov. and Deinococcus murrayi sp. nov., two extremely radiation resistant and slightly thermophilic species from hot springs. Int. J. Syst. Bacteriol. 47:939-947] at 47° C. with shaking for 40 h. The cells were harvested by centrifugation at 10,000×g for 10 min at 4° C. Genomic DNA was extracted from the cells using Puregene™ DNA purification kit (Gentra systems, Minneapolis, Minn.).

TABLE 1
Bacteria and plasmids

| | | Reference or |
|---|---|---|
| Bacteria or plasmids | Genotype and description | source |
| Bacteria | | |
| E. coli XL-1 blue | Host for regular cloning; | Stratagene |
| | recA1 endA1 gyrA96 thi-1 hsdR17 supE44 relA1 | |
| | lac [F′ proAB lacIqZΔM15 Tn10 (Tetr)] | |
| E. coli JM109 | Host for expression of carotenoid biosynthetic | Yanish- |
| | genes; | Perron¹ |
| | e14⁻(McrA⁻) recA1 endA1 gyrA96 thi-1 hsdR17 | |
| | (rK⁻ mK⁺) | |
| | supE44 relA1 Δ(lac-proAB) [F′ traD36 proAB | |
| | lacIqZΔM15] | |
| E. coli EPI100 | A fosmid clone containing a genomic DNA | Provided by |
| (RF001-12 M21) | fragment of Rhodococcus sp. RHA1 | W. W. Mohn² |
| Deinococcus | Type strain of Deinococcus geothermalis | DSMZ³ |
| geothermalis | | |
| DSM11300 | | |
| Plasmids | | |
| pUCmod | Vector for constitutive expression of carotenoid | Schmidt- |
| | biosynthetic genes; ampicillin and carbenicillin | Dannert⁴ |
| | resistant | |
| pACmod-EBI₁₄ | Vector containing crtE, crtB, and crtI₁₄ for | Schmidt- |
| | lycopene synthesis in E. coli; chloramphenicol | Dannert⁴ |
| | resistant | |
| pCR2.1-TOPO | Vector for direct TA-cloning of PCR product | Invitrogen |
| pUCmod-DgcrtLm- | D. geothermalis crtLm (1353 bp) in pUCmod | This study |
| F1-G | | |
| pUCmod-DgcrtLm- | D. geothermalis crtLm (1308 bp) in pUCmod | This study |
| F2-A | | |
| pTA-766-14 | Strain RHA1 crtLm (1293 bp) in pCR2.1-TOPO | This study |
| pUCmod-Rha766-2 | Strain RHA1 crtLm (1293 bp) and part of | This study |
| | pCR2.1-TOPO in pUCmod; 2 point mutations in | |
| | crtLm. | |
| pUCmod-766-SDM4 | Strain RHA1 crtLm (1293 bp) and part of | This study |
| | pCR2.1-TOPO in pUCmod; no mutation | |
| pUCmod-766-8 | Strain RHA1 crtLm (1293 bp) in pUCmod | This study |
| pUCmod-766-F2-6 | Strain RHA1 crtLm (1191 bp) in pUCmod | This study |
| pUCmod-766-F3-2 | Strain RHA1 crtLm (1149 bp) in pUCmod | This study |

¹Yanish-Perron C., Vieira J. and Messing J. (1985) Improved M13 phage cloning vectors and host strains: nucleotide sequences of the M13mp18 and pUC19 vectors. Gene 33: 103-119
²Mohn W. W. et. al. (2006) The complete genome of Rhodococcus sp. RHA1 provides insights into a catabolic powerhouse. PNAS 103: 15582-15587.
³German National Resource Centre for Biological Material
⁴Schmidt-Dannert C., Umeno D., and Arnold F. H. (2000) Molecular breeding of carotenoid biosynthetic pathway. Nat. Biotechnol. 18: 750-753

Cloning of Deinococcus geothermalis DSM 11300 lycopene β-monocyclase (crtLm) gene. The crtLm gene (also known as ORF2206) was amplified by PCR from D. geothermalis DSM 11300 genomic DNA with either primer Dg-crtLm-F1 or Dg-crtLm-F2 plus Dg-crtLm-R2 (Table 2) using Epicentre FailSafe™ PCR buffer L and FailSafe™ PCR enzyme mix. The PCR thermal profile was: (i) 5 cycles of 30 s at 95° C., 30 s at 55° C., and 90 s at 72° C., (ii) 25 cycles of 30 s at 95° C., 30 s at 60° C., and 90 s at 72° C., (iii) a 10 min soak at 72° C. and a hold at 4° C. Dg-crtLm-F1 and Dg-crtLm-F2 primers contained at the 5′ end a XbaI site followed by the Shine-Dalgarno sequence (AGGAGG) and a start codon (ATG). Dg-crtLm-R2 primer contained at its 5′ end an NcoI site. The Dg-crtLm-F1/R2 and Dg-crtLm-F2/R2 PCR products were digested with XbaI and NcoI, followed by ligation with plasmid pUCmod that was previously digested by XbaI and NcoI. The resulting plasmids were pUCmod-DgcrtLm-F1-G and pUCmod-DgcrtLm-F2-A. The presence of a correct insert in both plasmids was confirmed by DNA sequencing using primers pUCmod-F and pUCmod-R (Table 2).

Cloning of Rhodococcus sp. RHA1 lycopene β-monocyclase (crtLm) gene. The crtLm gene (also known as ORF766) of strain RHA1 was directly amplified from a single colony of E. coli EPI100(RF001-12 M21) with primers Rha766-F and Rha766-R (Table 2) by 30 cycles of PCR using Taq DNA polymerase with a thermal profile of 30 s at 95° C., 30 s at 58° C. and 60 s at 72° C., followed by a 10 min soak at 72° C. and a hold at 4° C. The PCR product was directly cloned into pCR2.1-TOPO, resulting in plasmid pTA-766-14. Plasmid pTA-766-14 was digested with XbaI and AseI. The 1.6-kb fragment was gel purified and ligated with plasmid pUCmod that was previously digested by XbaI and NdeI. The resulting plasmid was pUCmod-Rha766-2. DNA sequencing of pUCmod-Rha766-2 using primers pUCmod-F and pUCmod-R (Table 2) identified 2 point mutations within the cloned ORF766. These two mutations were reverted to the wild-type DNA sequence using Quikchange® Multi Site-Directed Mutagenesis kit (Stratagene) with primers Rha-1 mut and Rha-2 mut (Table 2), and the resulting plasmid was pUCmod-766-SDM4.

ORF766 was re-amplified from pUCmod-766-SDM4 with primers New766-F and New766-R (Table 2) using Pfu DNA polymerase. The 30-cycle thermal profile was 30 s at 95° C., 30 s at 58° C. and 90 s at 72° C. New766-F contained at its 5′ end a XbaI site followed by the Shine-Dalgarno sequence (AGGAGG) and a start codon (ATG). New766-R primer contained at its 5′ end an NcoI site. The PCR product was digested with XbaI and NcoI, followed by ligation with plasmid pUCmod that was previously digested by XbaI and NcoI. The resulting plasmid was pUCmod-766-8. DNA sequencing confirmed the presence of a correct insert in pUCmod-766-8.

ORF766 with alternative start codon was amplified from pUCmod-766-8 with either primer New766-F2 or New766-F3 plus primer New766-R (Table 2) using Pfu DNA polymerase. Primers New766-F2 and New766-F3 contained the same XbaI site and Shine-Dalgarno sequence as New766-F. The two PCR products were digested with XbaI and NcoI, followed by ligation with plasmid pUCmod that was previously digested by XbaI and NcoI. The two resulting plasmids were pUCmod-766-F2-6 and pUCmod-766-F3-2. DNA sequencing confirmed the presence of correct inserts in these two plasmids.

TABLE 2
Oligonucleotide primers.

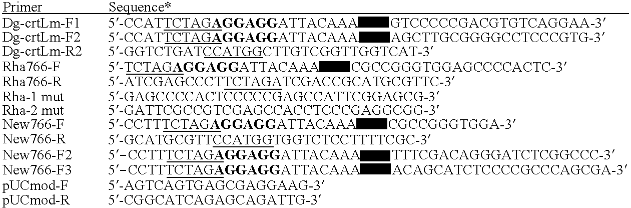

Restriction endonuclease sites are underlined. Bold face indicates the Shine-Dalgarno sequence. Start codons (ATG) are highlighted in black.

Cultivation of recombinant E. coli JM109 strains for carotenoid production. Plasmid pACmod-EBI₁₄ was mixed with various pUCmod derivatives that contained a lycopene β-monocyclase gene. Each DNA mixture was transformed into E. coli JM109 by electroporation and transformants were selected on LB media (Sambrook et al., 1989) containing 100 μg·mL⁻¹ ampicillin and 50 μg·mL⁻¹ chloramphenicol. A single colony from each transformation was used to inoculate 5 mL 2×YT broth (Sambrook et al., 1989) containing 100 μg·mL⁻¹ ampicillin and 50 μg·mL⁻¹ chloramphenicol. The broth culture was grown overnight at 37° C. with shaking at 230 rpm. The overnight seed culture was used to inoculate 150 to 200 mL 2×YT broth containing 100 μg·mL⁻¹ carbenicillin and 50 μg·mL⁻¹ chloramphenicol (in a 500-mL baffled-flask) to a cell density of 0.01 OD₆₀₀ unit. The culture was then cultivated in the dark for 48 h at 30° C. with shaking at 230 rpm.

Extraction and analysis of carotenoids. Fifty mL of cells were harvested by centrifugation at 10,000×g for 10 min at 4° C. The wet cells were extracted with 5 mL acetone for 10 min and the extracts were separated from the biomass by centrifugation at 10,000×g for 10 min at 4° C. The acetone extracts were kept at −80° C. for at least 1 h and a white precipitate would form. Precipitate-free acetone extracts (20 μL) were then injected into an Agilent HPLC system for carotenoid analysis using the following conditions: HPLC column is 25 cm×4.6 mm Micorsorb® C18 bonded silica gel, 100 Å pore size, and 5 um particle size (Varian, Inc.); mobile phase A is 90% acetonitrile, 10% methanol, mobile phase B is 45% hexanes, 45% methylene chloride, 9.9% methanol, and 0.1% diisopropylethylamine; a profile as set out in Table 3; flow rate of 0.70 ml/min; column temperature of 25° C.; detection at 454 nm; injection volume of 20 ul; and a stop time of 45 minutes (no post time).

TABLE 3
HPLC Mobile Phase Profile

| Time (min) | % Phase A | % Phase B |
|---|---|---|
| 0 | 95 | 5 |
| 10 | 95 | 5 |
| 40 | 45 | 55 |
| 41 | 95 | 5 |
| 45 | 95 | 5 |

The approximate retention times for peaks will be: Zeaxanthin 8.0 min; 3-OH-γ-carotene 18.0 min; β-cryptoxanthin 22.0 min; lycopene 25.8 min; torulene 27.1 min; γ-carotene 28.7 min; and β-carotene 32.0 min.

Results and Discussion

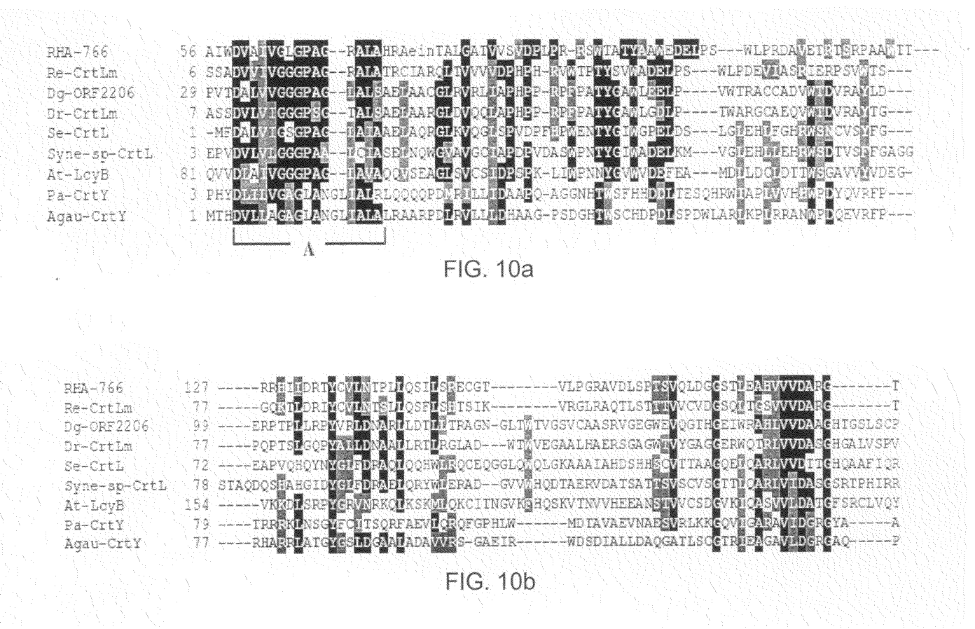

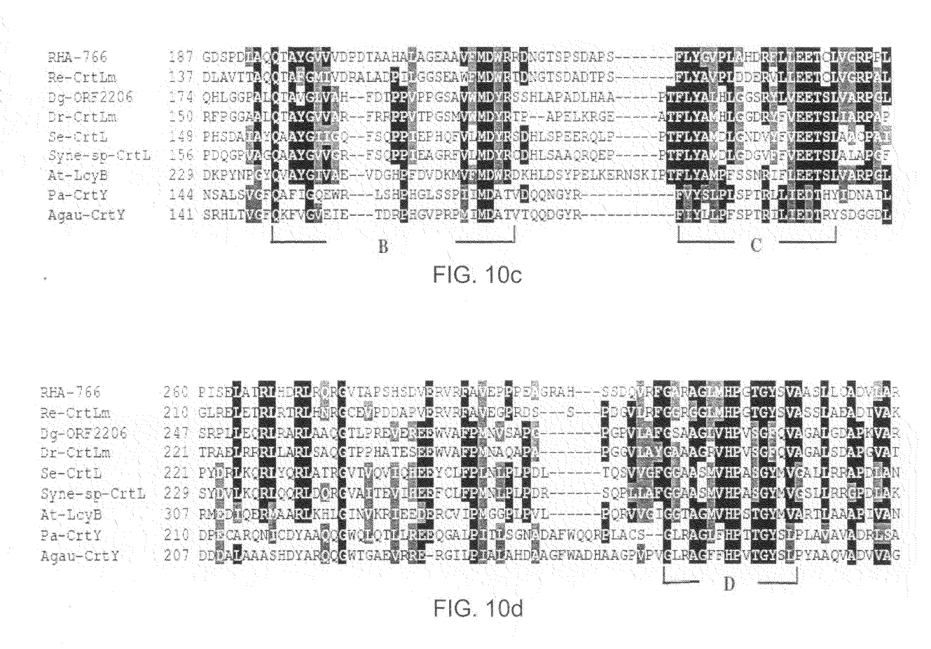

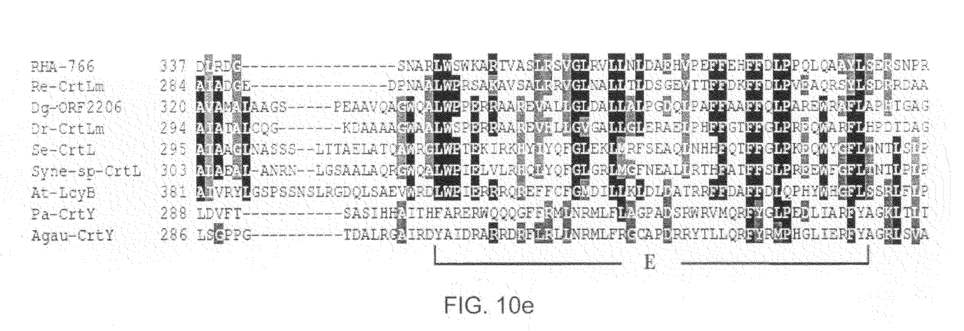

Identification and sequence analysis of putative lycopene β-monocyclase genes. The lycopene β-monocyclase genes, crtLm, of Rhodococcus erythropolis AN12 (Genbank accession no. AY437860) and Deinococcus radiodurans R1 (Genbank accession no. AAF10377.1) were used as queries to search current microbial genome sequencing project databases by TBLASTX [Altschul S. F., Madden T. L., Schaffer A. A., Zhang J., Zhang Z., Miller W., and Lipman D. J. (1997) Gapped BLAST and PSI-BLAST: a new generation of protein database search programs. Nucleic Acids Res. 25:3389-3402], which compares the six-frame translations of a nucleotide query sequence against the six-frame translations of a nucleotide sequence database. R. erythropolis crtLm showed highest homology to ORF766 in Rhodococcus sp. RHA1 genome which encodes a putative lycopene cyclase (SEQ ID 1). D. radiodurans crtLm showed highest homology to ORF2206 in Deinococcus geothermalis DSM11300 genome which also encodes a putative lycopene cyclase (SEQ ID 2). The deduced protein sequence of ORF766 and ORF2206 showed 52% and 61% identity, respectively, to R. erythropolis and D. radiodurans CrtLm protein sequences when the sequences were aligned by ClustalW. These two ORFs did not show significant homology to the lycopene β-monocyclase CrtYm identified in the marine bacterium strain P99-3 (Yanish-Perron, 1985). Interestingly, ORF766 and ORF2206 showed lower overall homology to the bacterial CrtY-type lycopene β-cyclases (˜20% identity) than to the CrtL-type lycopene β-cyclases found in the blue-green algae Synechococcus (˜25 to 36% identity) and the Lyc-b-type lycopene β-cyclases found in plants (˜25 to 30% identity). Although these two ORFs showed lower homology to the CrtY enzymes, the five conserved domains previously identified in the classical CrtY-type lycopene cyclases (29) are also present in ORF766, ORF2206, CrtLm of R. erythropolis and D. radiodurans, and CrtL of Synechococcus (FIG. 10), suggesting the CrtL-type and CrtY-type cyclases are evolutionary related. ORF766 of Rhodococcus sp. RHA1 and ORF2206 of D. geothermalis were putatively designated as crtLm due to their significant identities to well characterized crtLm genes.

Synthesis of γ-carotene in lycopene-producing E. coli upon expression of Rhodococcus sp. RHA1 ORF766. The function of ORF766 was examined by cloning the ORF into the expression plasmid pUCmod and expressing the ORF in lycopene-producing E. coli JM109 (pACmod-EBI₁₄). ORF766 was PCR amplified from E. coli EPI100 (RF001-12 M21) using primers Rha766-F and Rha766-R. XbaI sites were included in these two primers to facilitate direct cloning of the PCR-amplified ORF into the XbaI site of pUCmod. The PCR product was directly cloned into pCR2.1-TOPO and resulted in pTA-766-14. Plasmid pTA-766-14 was subjected to a series of restriction mapping experiment (data not shown) and it was concluded that the two XbaI sites located at both ends of the cloned PCR product could not be digested by XbaI. However, the PCR product was flanked by an XbaI site and an AseI site located on pCR2.1-TOPO. Restriction digestion of pTA-766-14 by XbaI and AseI released ORF766 plus a small segment of pCR2.1-TOPO from pTA-766-14 and allowed cloning of this piece of DNA into the XbaI-NdeI sites of pUCmod. The resultant plasmid was named as pUCmod-Rha766-2. DNA sequencing of pUCmod-Rha766-2 identified two point mutations within the cloned ORF766 which resulted in changes in the protein sequence. These two mutations were corrected by site-directed mutagenesis and the resulting plasmid was pUCmod-766-SDM4. The corrected ORF766 was re-cloned from pUCmod-766-SDM4 into pUCmod. High fidelity Pfu DNA polymerase was used to amplify ORF766 from pUCmod-766-SDM4 and this PCR product was cloned into the XbaI and NcoI site of pUCmod, resulting in pUCmod-766-8 (SEQ ID NO. 3).

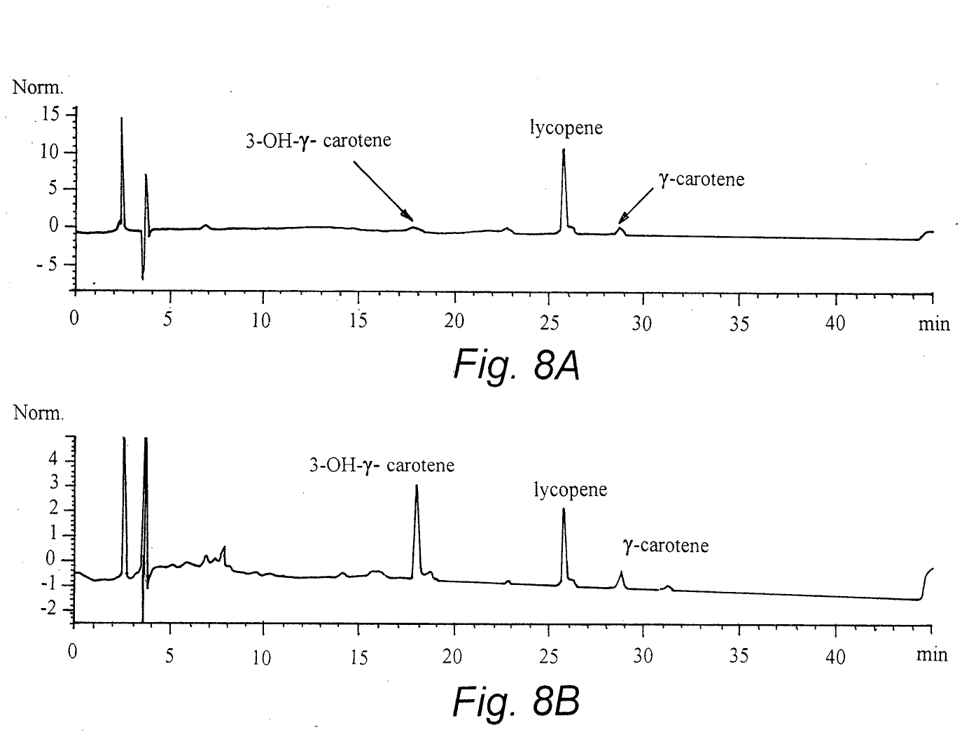

Cell extracts of E. coli JM109 (pACmod-EBI₁₄) and JM109 (pACmod-EBI₁₄/pUCmod-766-8) were analyzed by HPLC for carotenoid content. Lycopene was the only carotenoid detected in both extracts (FIGS. 8a and 8b). Two new forward primers, New766-F2 and New766-F3 (Table 2), were designed for PCR amplification of two shorter versions of ORF766. These two PCR products were cloned into the XbaI and NcoI site of pUCmod, resulting in pUCmod-766-F2-6 (SEQ ID NO. 4) and pUCmod-766-F3-2 (SEQ ID NO. 5).

HPLC analysis of JM109 (pACmod-EBI₁₄/pUCmod-766-F2-6) and JM109 (pACmod-EBI₁₄/pUCmod-766-F3-2) cell extracts identified three major metabolites (FIGS. 3c and 3d). One of the peaks was lycopene. The retention time of the second metabolite, Peak 1, was identical to that of an authentic γ-carotene standard (FIG. 3e). Additionally, the absorption maxima of Peak 1 (440, 463, and 495 nm) (FIG. 4a) were also identical to those of γ-carotene (FIG. 4b). The identity of the third metabolite, Peak 2, is currently not clear. However, this metabolite is not the bicyclic β-carotene because a β-carotene standard eluted from the HPLC system at about 32 min (FIG. 3f). Thus, the gene products of the two cloned ORF766 with an alternative start codon displayed lycopene β-monocyclase activities and transformed lycopene to γ-carotene. It is not presently clear which of the two start codons is the genuine start codon for ORF766 in vivo.

The HPLC peak shape of Peak 2 (FIGS. 3c and 3d) was asymmetrical, suggesting more than one compound was eluted from the HPLC column almost simultaneously. That the absorption spectrum of the earlier half of Peak 2 (FIG. 4c) was slightly different from that of the later half of Peak 2 (FIG. 4d) also supports co-elution of more than one compound. There is presently uncertainty as to the identity of the compound present in the later half of Peak 2. However, the unique absorption spectrum of the earlier half of Peak 2, with absorption maxima of 456 and 485, suggests this metabolite might be torulene. Pure torulene has absorption maxima of 460, 484, and 520 nm (in hexane) [Britton G., Liaaen-Jensen S., and Pfander H. (2004) Carotenoids handbook, Birkhäuser Verlag, Basel]. Torulene is not commercially available but if this compound was injected into the HPLC system used in this study, it is expected to elute from the C₁₈ column after lycopene but before γ-carotene and β-carotene based on similar HPLC analyses by Schmidt-Dannert et al. (2000). Torulene is the cyclization product of 3,4-didehyrolycopene. Schmidt-Dannert et al. (2000) showed that 3,4-didehyrolycopene was a metabolite of lycopene, produced by the gene product of crtI₁₄, a mutant lycopene desaturase located on pACmod-EBI₁₄ (Table 1). A wild-type lycopene desaturase (CrtI) sequentially introduces four double bonds into phytoene and produces lycopene. The mutant CrtI₁₄ enzyme, on the other hand, is capable of introducing up to six double bonds into phytoene and produces 3,4-didehydrolycopene and 3,4,3′,4′-tetradehydrolycopene [Schmidt-Dannert et al. (2000)]. The gene product of ORF766 might cyclize the Ψ-end of 3,4-didehydrolycopene and produced torulene. Alternatively, the mutant CrtI₁₄ protein might introduce an additional double bond into γ-carotene and produced torulene. Replacing the mutant crtI₁₄ with a wild-type crtI in the lycopene synthetic pathway may eliminate the formation of torulene and the unknown metabolite presence in the later half of Peak 2.

A study of carotenoid pigments from the genus Rhodococcus was carried out previously by Ichiyama et al. [Ichiyama S., Shimokata K., and Tsukamura M. (1989) Carotenoid pigments of genus Rhodococcus. Microbiol. Immunol. 33:503-508]. Based on that study, Rhodococcus species were classified into three groups: those which synthesize β-carotene, those which synthesize a γ-carotene-like substance, and those which produce neither carotene. Rhodococcus sp. RHA1 had not been shown to produce any carotenoid pigment under any growth conditions (Mohn W. W., Personal communication).

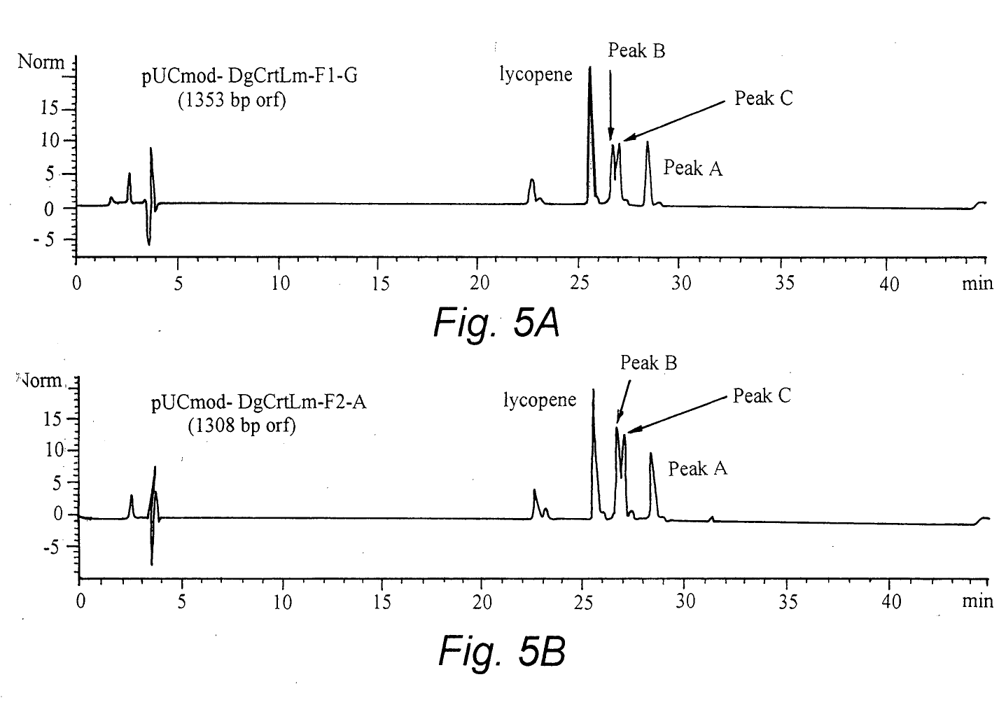

Synthesis of γ-carotene in a lycopene-producing E. coli upon expression of D. geothermalis ORF2206. The function of ORF2206 was examined by cloning the ORF into the expression plasmid pUCmod and expressing the ORF in lycopene-producing E. coli JM109 (pACmod-EBI₁₄). Two possible start codons for ORF2206 were annotated in D. geothermalis genome sequencing database, with the first possible start codon being 45 bp 5′ to the second possible start codon. Two different forward PCR primers, Dg-crtLm-F1 and Dg-crtLm-F2 (Table 2), were designed for PCR amplification of ORF2206 with the common reverse primer Dg-crtLm-R2 (Table 2). The ORF2206 amplified with primer Dg-crtLm-F1 was 45 bp longer than that amplified with primer Dg-crtLm-F2. These two versions of ORF2206 were cloned into pUCmod and resulted in plasmid pUCmod-DgcrtLm-F1-G (SEQ ID NO. 6) and pUCmod-DgcrtLm-F2-A (SEQ ID NO. 7). HPLC analysis of JM109 (pACmod-EBI₁₄/pUCmod-DgcrtLm-F1-G) and JM109 (pACmod-EBI₁₄/pUCmod-DgcrtLm-F2-A) cell extracts detected lycopene plus three additional metabolites (FIGS. 5a and 5b). Peak A had a retention time and absorption spectrum identical to that of an authentic γ-carotene standard (FIG. 3e) indicating this metabolite is γ-carotene. The bicyclic β-carotene was not detected in the cell extracts. Therefore, the gene product of ORF2206 displayed lycopene β-monocyclase activity which catalyzed monocyclization of lycopene to γ-carotene. Peak B and Peak C of FIG. 5 are possibly the two compounds observed in JM109 (pACmod-EBI₁₄/pUCmod-766-F2-6) and JM109 (pACmod-EBI₁₄/pUCmod-766-F3-2) cell extracts that were co-eluted as Peak 2 in FIGS. 3c and 3d. The absorption spectrum of Peak B was similar to that of the first half of Peak 2 (FIGS. 4c and 4d), suggesting it might be torulene.

Summary

Two new lycopene β-monocyclase genes (crtLm) were identified in Rhodococcus sp. RHA1 and Deinococcus geothermalis DSM11300. Their monocyclase activities were confirmed by expressing the genes in a lycopene-producing E. coli. Both gene products catalyzed the conversion of lycopene to γ-carotene without formation of the bicyclic β-carotene. The identification of these two new genes is a critical first step for developing an engineered β-cryptoxanthin synthetic pathway (FIG. 2).

### Example 2

Materials. All reagents were of the highest purity available and were purchased from Sigma (St. Louis, Mo.) Aldrich (Milwaukee, Wis.), and Fisher Scientific (Pittsburgh, Pa.) unless otherwise noted. Polymerase chain reaction (PCR) primers were purchased from Integrated DNA Technologies (Coralville, Iowa). Pfu DNA polymerase (Stratagene) was used in PCR reactions. Restriction endonucleases were purchased from Invitrogen (Carlsbad, Calif.), New England Biolabs (Beverly, Mass.), and Fermentas (Hanover, Mass.). Fast-Link™ DNA ligation kit was purchased from Epicentre (Madison, Wis.).

Bacterial strains and plasmids. The bacterial strains and plasmids used are listed in Table 4.

TABLE 4
Bacteria and plasmids.

| | | Reference or |
|---|---|---|
| Bacteria or plasmids | Genotype and description | source |
| Bacteria | | |
| E. coli XL-1 blue | Host for regular cloning; | Stratagene |
| | recA1 endA1 gyrA96 thi-1 hsdR17 supE44 relA1 lac | |
| | [F′ proAB lacIqZΔM15 Tn10 (Tetr)] | |
| E. coli JM109 | Host for expression of carotenoid biosynthetic genes; | Yanish et al.¹ |
| | e14⁻(McrA⁻) recA1 endA1 gyrA96 thi-1 hsdR17 | |
| | (rK⁻ mK⁺) | |
| | supE44 relA1 Δ(lac-proAB) [F′ traD36 proAB | |
| | lacIqZΔM15] | |
| Plasmids | | |
| pBBR1MCS-2 | Broad-host-range plasmid that is compatible with | Kovach et al.² |
| | derivatives of pUCmod and pACmod; kanamycin | |
| | resistant | |
| pBAD18 | Vector containing araC and the arabinose inducible | Guzman et al.³ |
| | P_{BAD} promoter; ampicillin resistant | |
| pUCmod | Vector for constitutive expression of carotenoid | Schnidt- |
| | biosynthetic genes; ampicillin and carbenicillin | Dannert et al.⁴ |
| | resistant | |
| pACmod-EBI₁₄ | Vector containing Pantoea ananatis wild type crtE | Schnidt- |
| | and crtB, plus P. ananatis mutant crtI₁₄ for lycopene | Dannert et al.⁴ |
| | synthesis in E. coli; chloramphenicol resistant | |
| pUCmod-766-F2-6 | Rhodococcus sp. RHA1 crtLm (1191 bp) in pUCmod | This study |
| pUCmod-DgcrtLm- | Deinococcus geothermalis crtLm (1308 bp) in | This study |
| F2-A | pUCmod | |
| pUCmod-Pa-crtZ | P. ananatis wild type crtZ in pUCmod | Prior |
| | | application⁵ |
| pUCmod-crtI | P. ananatis wild type crtI in pUCmod | This study |
| pACmod-EBI | Vector containing P. ananatis wild type crtE, crtB, | This study |
| | and crtI for lycopene synthesis in E. coli; | |
| | chloramphenicol resistant | |
| pACmod-EBIZ | Vector containing P. ananatis wild type crtE, crtB, | This study |
| | crtI, and crtZ; chloramphenicol resistant | |
| pBAD18-crtY | pBAD18 containing P. ananatis crtY regulated by the | This study |
| | arabinose inducible P_{BAD} promoter; ampicillin | |
| | resistant | |
| pBBR1-crtY | pBBR1MCS-2 containing araC and P. ananatis crtY | This study |
| | regulated by the P_{BAD} promoter; kanamycin resistant | |

¹Yanish-Perron C., Vieira J. and Messing J. (1985) Improved M13 phage cloning vectors and host strains: nucleotide sequences of the M13mp18 and pUC19 vectors. Gene 33: 103-119.
²Kovach M. E., Elzer P. H., Hill D. S., Robertson G. T., Farris M. A., Roop R. M., and Peterson K. M. (1995) Four new derivatives of the broad-host-range cloning vector pBBR1MCS, carrying different antibiotic-resistance cassettes. Gene 166: 175-176.
³Guzman L., Belin D. Carson M. J., and Beckwith J. (1995) Tight regulation, modulation, and high-level expression by vectors containing the arabinose P_{BAD} promoter. J. Bacteriol. 177: 4121-4130.
⁴Schmidt-Dannert C., Umeno D., and Arnold F. H. (2000) Molecular breeding of carotenoid biosynthetic pathway. Nat. Biotechnol. 18: 750-753.
⁵U.S. Pat. Appln. Serial No. 11/546,702, filed Oct. 12, 2006.

Cloning of Pantoea ananatis phytoene dehydrogenase (crtI) gene. The crtI gene was amplified by PCR from Pantoea ananatis genomic DNA (ATCC 19321D) with primers crtI-F plus crtI-R (Table 5) using Pfu DNA polymerase. The PCR thermal profile was: 30 cycles of 30 at 95° C., 30 s at 58° C., and 90 s at 72° C., followed by a 10 min soak at 72° C. and a hold at 4° C. Primer crtI-F contained at the 5′ end an XbaI site followed by the Shine-Dalgarno sequence (AGGAGG) and a start codon (ATG). Primer crtI-R contained at its 5′ end an NcoI site. The crtI PCR product was digested with XbaI and NcoI, followed by ligation with plasmid pUCmod that was previously digested by XbaI and NcoI. DNA sequencing of the resultant plasmid, using primers pUCmod-F and pUCmod-R (Table 4), revealed 2 mutations in the PCR-amplified crtI gene. The first mutation was a single nucleotide deletion that was 3′ to the ATG start codon of crtI. The second mutation was a silent mutation that did not result in any change in the protein sequence. Therefore, only the first mutation was corrected by using the QuikChange® Site-Directed Mutagenesis Kit (Stratagene) with primers crtI-mutF and crtI-mutR (Table 4). The resultant plasmid was named pUCmod-crtI.

The modified-lac promoter-crtI (P_{lac mod}-crtI) cassette was amplified from pUCmod-crtI using primers Plac-crtI-F and Plac-crtI-R. A HindIII site existed at the 5′ ends of both primers. Plasmid pACmod-EBI₁₄ was digested with HindIII and the pACmod-EB fragment was purified. This fragment was ligated to HindIII-digested P_{lac mod}-crtI to form plasmid pACmod-EBI.

Generating plasmid pACmod-EBIZ. The P_{lac mod}-crtZ cassette was amplified from pUCmod-Pa-crtZ using primers Plac-766-F and Plac-766-R (Table 5). The PCR thermal profile was: 30 cycles of 30 s at 95° C., 30 s at 55° C., and 1 min at 72° C., followed by a 10 min soak at 72° C. and a hold at 4° C. A PpuMI site existed at the 5′ ends of both primers. The PCR product was digested with PpuMI and ligated with plasmid pACmod-EBI that was previously digested with PpuMI. The resultant plasmid was pACmod-EBIZ.

Cloning of Pantoea ananatis β-carotene cyclase (crtY) gene. The crtY gene was amplified by PCR from pUCmod-crtY with primers pBAD-Y-F plus pBAD-Y—R (Table 5) using Pfu DNA polymerase. The PCR thermal profile was: 30 cycles of 30 s at 95° C., 30 s at 52° C., and 72 s at 72° C., followed by a 10 min soak at 72° C. and a hold at 4° C. Primer pBAD-Y-F hybridized to a region on pUCmod-crtY that was 5′ to an EcoRI site, a Shine-Dalgarno sequence (AGGAGG), and crtY start codon. Primer pBAD-Y—R contained at its 5′ end an XbaI site. The PCR product was digested with EcoRI and XbaI, followed by ligation with plasmid pBAD18, which was previously digested by EcoRI and XbaI, and formed pBAD-crtY. pBAD-crtY was then digested with ClaI, AccI, and PstI to generate 4 fragments of 2.5, 1.8, 1.0, and 0.4 kb. The 2.5-kb AccI-ClaI fragment that contained the araC-P_{BAD}-crtY cassette was gel-purified and ligated to a 4.1-kb AsuII fragment of pBBR1MCS-2 that contained a broad-host-range origin of replication and a gene coding for a kanamycin resistant determinant. The resultant plasmid was pBBR1-crtY.

TABLE 5
Oligonucleotide primers.

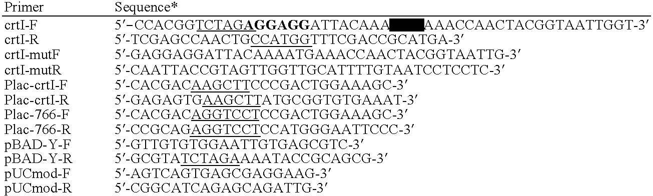

*Restriction endonuclease sites are underlined. Bold face indicates the Shine-Dalgarno sequence. Start codons (ATG) are highlighted in black.

Cultivation of recombinant E. coli JM109 strains for carotenoid production. Carotenogenic plasmids were transformed into E. coli JM109 by electroporation and transformants were selected on LB media [Sambrook, J., Fritsch, E. F., and Maniatis, T. (1989) Molecular cloning: a laboratory manual, 2nd Ed., Cold Spring Harbor laboratory Press, Cold Spring Harbor, N.Y.] containing the appropriate antibiotics (ampicillin at 100 μg·mL⁻¹, chloramphenicol at 50 μg·mL⁻¹, and kanamycin at 30 μg·mL⁻¹). A single colony from each transformation was used to inoculate 5 mL 2×YT broth [Sambrook et al. (1989)] containing the required antibiotics. The culture was grown overnight at 37° C. with shaking at 230 rpm. The overnight seed culture was used to inoculate 150 to 200 mL 2×YT broth (in a 500-mL baffled-flask), with antibiotics, to a cell density of 0.01 OD₆₀₀ unit. The culture was then cultivated in the dark for 24 to 48 h at 30° C. with shaking at 230 rpm.

JM109 (pACmod-EBIZ/pUCmod-766-F2-6/pBBR1-crtY) was cultivated as described above with the following modifications. An overnight seed culture was used to inoculate 200 mL 2×YT broth (in a 500-mL baffled-flask), with antibiotics, to a cell density of 0.01 OD₆₀₀ unit. The culture was then cultivated in the dark for 24 h at 30° C. with shaking at 230 rpm. The cells were then divided into 2 100-mL aliquots and harvested by centrifugation at 10,000×g for 5 min at room temperature. One cell pellet was immediately stored at −80° C. until carotenoid extraction. The second cell pellet was re-suspended with 100 mL 2×YT broth with antibiotics plus 0.2% (w/v) L-arabinose. The cell suspension was incubated without shaking inside an anaerobic gas jar, equipped with a GasPak Plus™ anaerobic system envelope (Fisher), for 6 hours at 30° C. After that, the cells were harvested for carotenoid extraction.

Extraction and analysis of carotenoids. Cells were harvested by centrifugation at 10,000×g for 10 min at 4° C. The wet cells were extracted with 5 mL acetone for 10 min and the extracts were separated from the biomass by centrifugation at 10,000×g for 10 min at 4° C. The acetone extracts were kept at −80° C. for at least 1 h and a white precipitate would form. Precipitate-free acetone extracts (at least 20 μL) were then injected into an Agilent HPLC system for carotenoid analysis using the following conditions: HPLC column is 25 cm×4.6 mm Micorsorb® C18 bonded silica gel, 100 Å pore size, and 5 um particle size (Varian, Inc.); mobile phase A is 90% acetonitrile, 10% methanol, mobile phase B is 45% hexanes, 45% methylene chloride, 9.9% methanol, and 0.1% diisopropylethylamine; a profile as set out in Table 6; flow rate of 0.70 ml/min; column temperature of 25° C.; detection at 454 nm; injection volume of 20 ul; and a stop time of 45 minutes (no post time).

TABLE 6
HPLC Mobile Phase Profile

| Time (min) | % Phase A | % Phase B |
|---|---|---|
| 0 | 95 | 5 |
| 10 | 95 | 5 |
| 40 | 45 | 55 |
| 41 | 95 | 5 |
| 45 | 95 | 5 |

The approximate retention times for peaks will be: Zeaxanthin 8.0 min; 3-OH-γ-carotene 18.0 min; β-cryptoxanthin 22.0 min; lycopene 25.8 min; torulene 27.1 min; γ-carotene 28.7 min; and β-carotene 32.0 min.

Results and Discussion

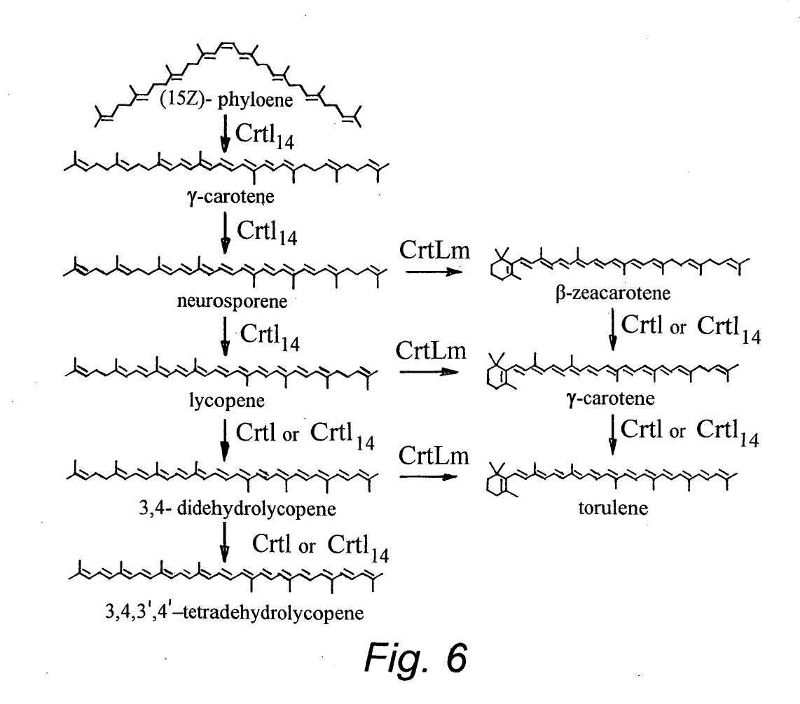

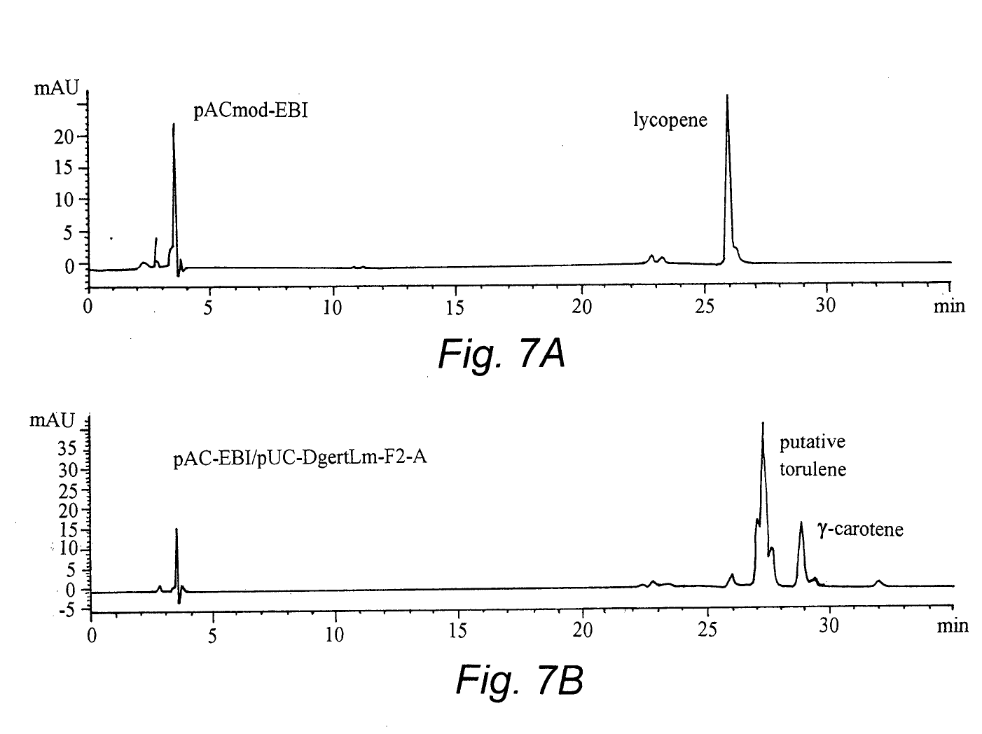

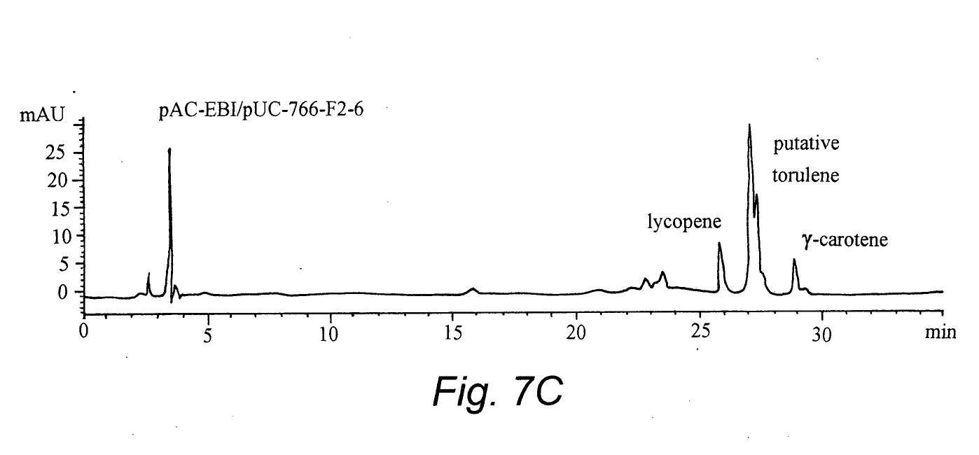

Replacement of a mutant crtI₁₄ gene with a wild type crtI gene did not eliminate the formation of torulene. In the previous example, it was shown that cloning a lycopene β-monocyclase gene (crtLm) into lycopene-producing E. coli JM109 (pACmod-EBI₁₄) resulted in the formation of γ-carotene plus an unknown metabolite. This metabolite was speculated to be torulene based on its absorption spectrum and its retention time. The mutant crtI₁₄ (phytoene dehydrogenase) gene was hypothesized to be the reason why torulene was synthesized in the presence of a crtLm gene [Schmidt-Dannert C., Umeno D., and Arnold F. H. (2000) Molecular breeding of carotenoid biosynthetic pathway. Nat. Biotechnol. 18:750-753]. FIG. 6 is a drawing of a metabolic pathway that explains the production of torulene. CrtI₁₄, a 6-step mutated phytoene dehydrogenase, produces 3,4-didehydrolycopene which could be metabolized by CrtLm to form torulene. Alternatively, CrtI₁₄ could transform γ-carotene to torulene. Wild-type CrtI, normally a 4-step phytoene dehydrogenase, was reported to have weak lycopene-oxidizing activities (red arrow) [Linden H., Misawa N., Chamovitz D. Pecker I., Hirschberg J., and Sandmann G. (1991) Functional complementation in Escherichia coli of different phytoene desaturase genes and analysis of accumulated carotenes. Z. Naturoforsch. 46C: 1045-1051; Fraser P. D., Misawa N., Linden H., Yamano S., Kobayashi K., and Sandmann G. (1992) Expression in Escherichia coli, purification, and reactivation of the recombinant Erwinia uredovora phytoene desaturase. J. Biol. Chem. 267:19891-19895; Umeno D., Tobias A. V., and Arnold F. H. (2002) Evolution of the C₃₀ carotenoid synthase CrtM for function in a C₄₀ pathway. J. Bacteriol. 184:6690-6699], which supports our observation of the formation of γ-carotene when crtI and crtLm were present in the same organism. As a consequence, the mutant crtI₁₄ was replaced by a wild type crtI, and the carotenoid content was examined. Replacement of the mutant crtI₁₄ with wild-type crtI did not prevent formation of the putative torulene (FIG. 7). The results were surprising because crtI of Pantoea ananatis was normally classified as a 4-step phytoene dehydrogenase, which oxidized phytoene to lycopene. However, production of dehydrolycopene by CrtI both in vivo and in vitro has been reported [Linden et al. (1991); Fraser et al. (1992)]. Umeno et al. (2002) also recently showed the 5-10% of total carotenoids in E. coli XL1-Blue (pUC-crtE-crtB-crtI) being 3,4,3′,4′-tetradehydrolycopene, which supports the feasibility of the wild-type crtI and crtLm to form torulene (FIG. 6).

Cloning of a crtZ into γ-carotene-producing E. coli led to the production of 3-OH-γ-carotene. Plasmids pUCmod-766-F2-6 and pUCmod-DgcrtLm-F2-A were independently transformed into JM109 (pACmod-EBIZ). These 2 plasmids respectively contained the crtLm genes of Rhodococcus sp. RHA1 and Deinococcus geothermalis. Cell extracts of JM109 (pACmod-EBIZ/pUCmod-DgcrtLm-F2-A) contained lycopene (FIG. 8A), a small amount of γ-carotene, and a new minor metabolite that was eluted from the C₁₈-reverse phase HPLC column at about 18 min using the method described above. This metabolite was more dominant in cell extracts of JM109 (pACmod-EBIZ/pUCmod-766-F2-6) (FIG. 8B). Torulene (retention time of 27.1 min) was not detected in these 2 cell extracts.

3-Hydroxy-γ-carotene and 3-hydroxy-torulene were expected to be produced in JM109 (pACmod-EBIZ/pUCmod-766-F2-6 or pUCmod-DgcrtLm-F2-A) because the crtZ gene product was expected to introduce a hydroxyl group into the β-ionone ring of γ-carotene and torulene. However, only one new metabolite, with a retention time of 18 min, was detected in the cell extracts. The fact that this metabolite was eluted from the C₁₈-reverse phase HPLC column earlier than lycopene, torulene, or γ-carotene suggests that it is more hydrophilic than the former compounds and could possibly be the hydroxylated form of torulene or γ-carotene. This metabolite had absorption maxima at 438, 464, and 495 nm, which is almost identical to the absorption spectra of γ-carotene and 3-hydroxy-γ-carotene, and is significantly different from the absorption spectra of torulene and 3-hydroxy-torulene [Mercadante A. Z., Steck A., and Pfander H. (1999) Carotenoids from Guava (Psidium guajava L.): Isolation and structure elucidation. J. Agric. Food Chem. 47:145-151; Britton G., Liaaen-Jensen S., and Pfander H. (2004) Carotenoids handbook, Birkhäuser Verlag, Basel]. Therefore, it is highly likely that this metabolite, with a retention time of about 18 min, is 3-hydroxy-γ-carotene. It is surprising that cloning crtZ into γ-carotene-producing E. coli reduced the production of torulene. One possibility is that CrtZ had a much higher affinity for γ-carotene than CrtI's affinity for either lycopene or γ-carotene and this resulted in pulling most of the carbon flux down the lycopene→γ-carotene→3-hydroxy-γ-carotene pathway (FIG. 6). An alternative substrate for β-monocyclase is neurosporene. β-monocyclase may compete with neurosporene hydroxylase to convert neurosporene to β-zeacarotene. Then β-zeacarotene may be hydroxylated to γ-carotene for further conversion to 3-OH-γ-carotene and then β-cryptoxanthin in the described pathway (FIGS. 2 and 6).

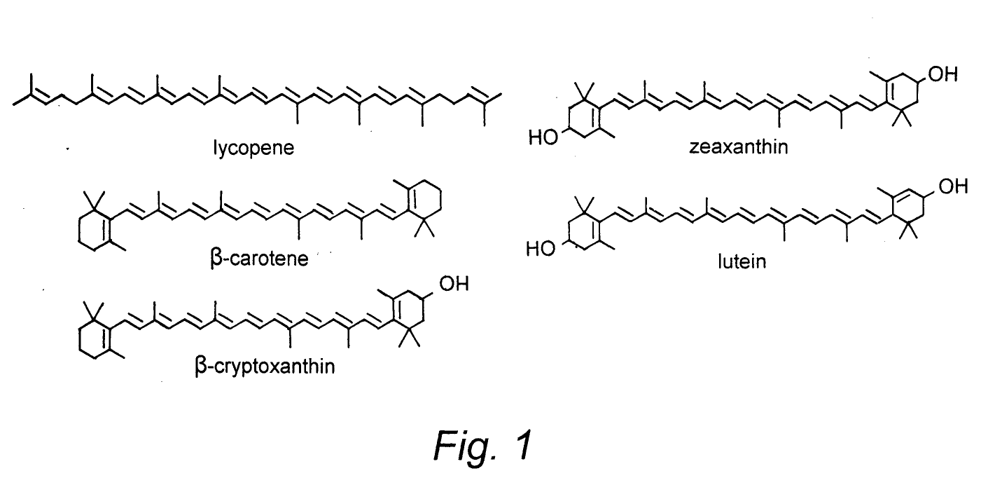

Cloning of an inducible P. ananatis crtY into JM109 (pACmod-EBIZ/pUCmod-766-F2-6). In order to convert 3-hydroxy-γ-carotene to BCX, a lycopene β-bicyclase from P. ananatis, crtY, was cloned into JM109 (pACmod-EBIZ/pUCmod-766-F2-6). The crtY gene was present on plasmid pBBR1-crtY, regulated by the P_{BAD} promoter of the E. coli arabinose operon. The P_{BAD} promoter regulatory gene, araC, is also present on pBBR1-crtY. The P_{BAD}-araC system offers regulatable control of gene expression in the presence of arabinose (iinducer) and tight control in the absence of inducer [Guzman L., Belin D. Carson M. J., and Beckwith J. (1995) Tight regulation, modulation, and high-level expression by vectors containing the arabinose P_{BAD} promoter. J. Bacteriol. 177:4121-4130]. A tightly regulated crtY is a critical element of this engineered BCX synthetic pathway (FIG. 1). This is because the remaining carotenogenic genes in this engineered pathway (crtE, crtB, crtI, crtZ, and crtLm) were constitutively expressed. High-level leaky expression of crtY would result in formation of β-carotene since CrtY and CrtLm would compete with each other for lycopene. Any β-carotene formed would then be converted by CrtZ to zeaxanthin. The yield of β-cryptoxanthin would be reduced by this side reaction as some lycopene would be used to synthesize β-carotene and zeaxanthin. The second critical element of this engineered BCX pathway is that after induction of crtY, the cells should be incubated in an environment with limited supply of oxygen. This is because cyclization of 3-hydroxy-γ-carotene by CrtY would produce β-cryptoxanthin, which is a substrate for CrtZ (crtZ is constitutively expressed inside the cells). Limiting the supply of oxygen to the cells after induction of crtY should minimize CrtZ activities as CrtZ activity is strictly oxygen-requiring [Fraser P. D., Miura Y., and Misawa N. (1997) In vitro characterization of astaxanthin biosynthetic enzymes. J. Biol. Chem. 272:6128-6135].

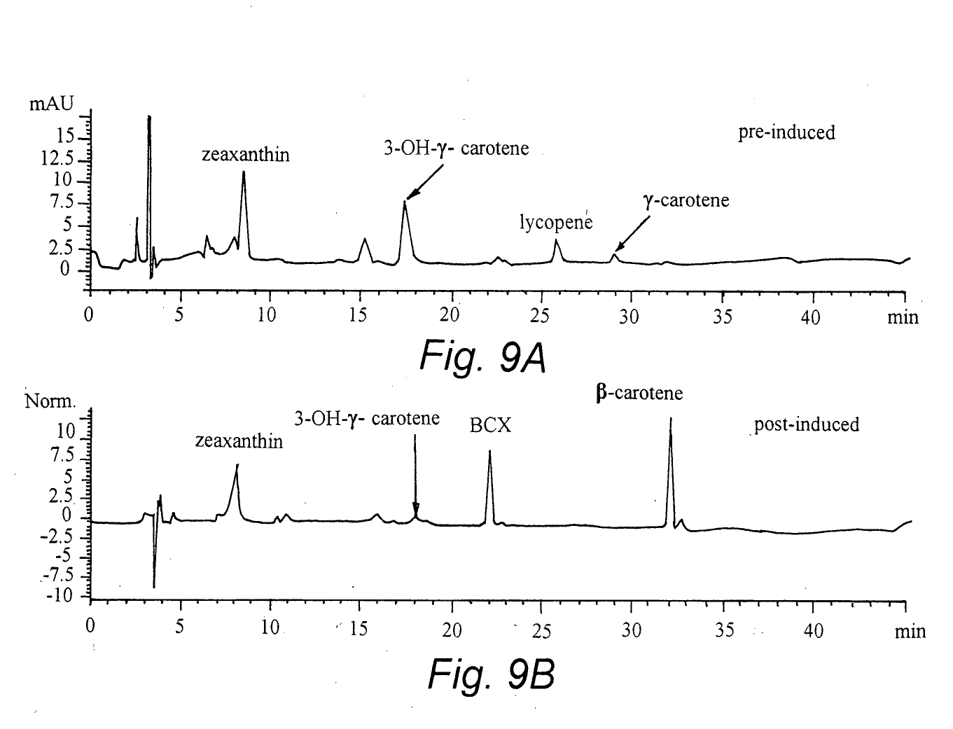

Carotenoids were extracted from cells before and after induction by L-arabinose. 3-Hydroxy-γ-carotene and zeaxanthin were detected in the pre-induced cell extracts, suggesting there was leaky-expression of crtY (FIG. 9A). After induction for 6 hours in an anoxic environment, 3-hydroxy-γ-carotene disappeared while β-cryptoxanthin was formed (FIG. 9B). β-carotene was also detected in the post-induction cell extracts, probably due to constitutive expression of the crtEBI after crtY was induced. The data demonstrate the feasibility of synthesizing β-cryptoxanthin using a lycopene β-monocyclase to convert lycopene to γ-carotene. FIG. 2 illustrates an engineered metabolic pathway that uses a lycopene β-monocyclase to convert lycopene to γ-carotene. γ-carotene is further hydroxylated by a β-carotene hydroxylase (CrtZ) to form 3-OH-γ-carotene. Finally, an inducible lycopene β-bicyclase (CrtY) is used to cyclize the Ψ-end (shaded in gray) of 3-OH-γ-carotene and results in β-cryptoxanthin.

Summary

β-cryptoxanthin was successfully synthesized in a recombinant E. coli that contained a lycopene β-monocyclase gene. The lycopene β-monocyclase gene is a critical component of an engineered metabolic pathway that converted lycopene to β-cryptoxanthin through γ-carotene and 3-hydroxy-γ-carotene.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

1. A method for the production of β-cryptoxanthin, comprising the steps of:
(a) providing a host cell which produces lycopene or neurosporene wherein said host cell expresses a lycopene β-monocyclase that converts lycopene or neurosporene into γ-carotene or β-zeacarotene, a lycopene hydroxylase that hydroxylates γ-carotene to 3-hydroxy-γ-carotene and β-zeacarotene to 3-hydroxy-β-zeacarotene, and an incucible lycopene β-bicyclase that converts 3-hydroxy-γ-carotene to β-cryptoxanthin;
(b) growing the host cell under conditions whereby γ-carotene is produced and is hydroxylated to 3-hydroxy-γ-carotene and which is converted into β-cryptoxanthin; and
(c) optionally recovering the β-cryptoxanthin.

2. A method according to claim 1 wherein the host cell is selected from the group consisting of bacteria, yeast, fungi, algae, and green plants.

3. A method according to claim 2, wherein the host cell is selected from the group consisting of carotenoid or isoprenoid producing bacteria and carotenoid or isoprenoid producing fungi.

4. A method according to claim 2, wherein the host cell is selected from the group consisting of Acinetobacter, Agrobacterium, Alcaligenes, Anabaena, Aspergillus, Bacillus, Brevibacterium, Candida, Chlorobium, Chromatium, Corynecbacteria, Cytophaga, Deinococcus, Erwinia, Erythrobacter, Eshcerichia, Flavobacterium, Hansenula, Klebsiella, Methanobacterium, Methylobacter, Methyloccocus, Methylocystis, Methylomicrobium, Methylomonas, Methylsinus, Mycobacterium, Myxococcus, Pantoea, Pichia, Pseudomonas, Rhodobacter, Rhodococcus, Saccharomyces, Salmonella, Sphingomonas, Streptomyces, Synechococcus, Synechocystis, Thiobacillus, Trichodenna, and Zymomonas.

5. A method according to claim 2, wherein the green plant is selected from the group consisting of alfalfa, Arabidopsis, barley, carrot, corn, oats, pepper, pumpkin, rice, sorghum, soybeans, and wheat.

6. (canceled)

7. (canceled)

8. (canceled)

9. (canceled)

10. (canceled)

11. (canceled)

12. An isolated nucleic acid molecule, comprising a nucleic acid molecule having the nucleic acid sequence selected from the group consisting of SEQ ID NO. 1 and SEQ ID NO.2.

13. An isolated nucleic acid molecule, comprising a nucleic acid sequence that encodes an enzyme having lycopene β-monocyclase activity, wherein said enzyme has an amino acid sequence encoded by a nucleotide sequence which hybridizes to SEQ ID NO. 1 or SEQ ID NO. 2 under stringency conditions represented by a final wash of 30 minutes in 0.1×SSC, 0.1% SDS at a temperature of 65° C.

14. An isolated nucleic acid molecule, comprising a nucleic acid molecule that encodes a polypeptide having 65% or more homology to a polypeptide selected from the group consisting of SEQ ID NO. 1 and SEQ ID NO. 2 as measured by the ClustalW method or the Smith-Waterman method.

15. An isolated nucleic acid molecule as defined in claim 14, wherein the polypeptide has 80% or more homology to SEQ ID NO. 1 or SEQ ID NO. 2.

16. An isolated nucleic acid molecule as defined in claim 15, wherein the polypeptide has 90% or more homology to SEQ ID NO. 1 or SEQ ID NO. 2.


SEQUENCE LISTING (7 sequences)

SEQ ID NO: 1 - DNA, 1293 bp, Rhodococcus sp.
gtgcgccgggtggagccccactcccccgagccattcggagcgtacgacgatgagttcggtaaatattgtagttccgtccgggtccgactttggaacggaggcttgtttcgacagggatctcggcccccgtgtcaggcttgctgcgtgacagcatctccccgcccagcgatctgggacgttgcgatcgtgggcctcgggcctgccggccgcgccctggcacacagggccacggctctcggtgcgacggtcgtctcggtcgacccactcccccgcagatcgtggaccgcgacgtacgcggcgtgggaagacgagctccccagctggctcccgagggacgcggtcgaaacgagaacttctcgcccggcagcctggacgacacgtcgccacatcatcgatcgcacctattgtgttttgaatacacccttactgcaatcaatactcagtagggaatgtggaactgttctcccaggcagagccgtggacctctcccccacctccgtccagctggacggagggtccacgctggaggcgcatgtcgtcgtcgacgcccgcggcaccggcgacagccccgacctcgcccagcagacggcatacggcgtcgtggtggaccccgacacggccgcacatgcgctcgccggcgaggccgcggtgttcatggactggcgccgcgacaacggcacatcaccctcggacgcgccgtccttcctctacggggttccgctcgcgcacgaccggttcctgctcgaagagacctgcctggtcggtcgcccgccactcccgatctcggaactcgccacccgactccacgaccgactccgacagcggggcgtcaccgcgccgtcccattcggacgtcgagagagtcagattcgccgtcgagccacctcccgaggcgggtcgtgcccacagctcggatcaggtgcgcttcggggcccgcgccgggttgatgcaccccggaacgggctacagcgtcgcagcgtcgctcctgtgcgcagatgttctagcccgcgatcttcgtgacggttcgaacgcgcgactgtggtcgtggaaggcccgcaccgtcgcgtccctgcggtcggttggtctccgcgtcctcctgaatctggatgcggaacacgtccccgaattcttcgagcatttcttcgatctgccgccacaactccaggcggcatatctatctgaacgatcgaatccgcgcggcaccctgtcggcaatggggcggatgtttcgggcatcctccggcgatatccgcagtgtgattctccgttcgacgcttcgccgggaagttcgatga

SEQ ID NO: 2 - DNA, 1308 bp, Deinococcus sp. [sequence omitted]

SEQ ID NO: 3 - DNA, 1293 bp, Rhodococcus sp.
atgcgccgggtggagccccactcccccgagccattcggagcgtacgacgatgagttcggtaaatattgtagttccgtccgggtccgactttggaacggaggcttgtttcgacagggatctcggcccccgtgtcaggcttgctgcgtgacagcatctccccgcccagcgatctgggacgttgcgatcgtgggcctcgggcctgccggccgcgccctggcacacagggccacggctctcggtgcgacggtcgtctcggtcgacccactcccccgcagatcgtggaccgcgacgtacgcggcgtgggaagacgagctccccagctggctcccgagggacgcggtcgaaacgagaacttctcgcccggcagcctggacgacacgtcgccacatcatcgatcgcacctattgtgttttgaatacacccttactgcaatcaatactcagtagggaatgtggaactgttctcccaggcagagccgtggacctctcccccacctccgtccagctggacggagggtccacgctggaggcgcatgtcgtcgtcgacgcccgcggcaccggcgacagccccgacctcgcccagcagacggcatacggcgtcgtggtggaccccgacacggccgcacatgcgctcgccggcgaggccgcggtgttcatggactggcgccgcgacaacggcacatcaccctcggacgcgccgtccttcctctacggggttccgctcgcgcacgaccggttcctgctcgaagagacctgcctggtcggtcgcccgccactcccgatctcggaactcgccacccgactccacgaccgactccgacagcggggcgtcaccgcgccgtcccattcggacgtcgagagagtcagattcgccgtcgagccacctcccgaggcgggtcgtgcccacagctcggatcaggtgcgcttcggggcccgcgccgggttgatgcaccccggaacgggctacagcgtcgcagcgtcgctcctgtgcgcagatgttctagcccgcgatcttcgtgacggttcgaacgcgcgactgtggtcgtggaaggcccgcaccgtcgcgtccctgcggtcggttggtctccgcgtcctcctgaatctggatgcggaacacgtccccgaattcttcgagcatttcttcgatctgccgccacaactccaggcggcatatctatctgaacgatcgaatccgcgcggcaccctgtcggcaatggggcggatgtttcgggcatcctccggcgatatccgcagtgtgattctccgttcgacgcttcgccgggaagttcgatga

SEQ ID NO: 4 - DNA, 1149 bp, Rhodococcus sp.
atgacagcatctccccgcccagcgatctgggacgttgcgatcgtgggcctcgggcctgccggccgcgccctggcacacagggccacggctctcggtgcgacggtcgtctcggtcgacccactcccccgcagatcgtggaccgcgacgtacgcggcgtgggaagacgagctccccagctggctcccgagggacgcggtcgaaacgagaacttctcgcccggcagcctggacgacacgtcgccacatcatcgatcgcacctattgtgttttgaatacacccttactgcaatcaatactcagtagggaatgtggaactgttctcccaggcagagccgtggacctctcccccacctccgtccagctggacggagggtccacgctggaggcgcatgtcgtcgtcgacgcccgcggcaccggcgacagccccgacctcgcccagcagacggcatacggcgtcgtggtggaccccgacacggccgcacatgcgctcgccggcgaggccgcggtgttcatggactggcgccgcgacaacggcacatcaccctcggacgcgccgtccttcctctacggggttccgctcgcgcacgaccggttcctgctcgaagagacctgcctggtcggtcgcccgccactcccgatctcggaactcgccacccgactccacgaccgactccgacagcggggcgtcaccgcgccgtcccattcggacgtcgagagagtcagattcgccgtcgagccacctcccgaggcgggtcgtgcccacagctcggatcaggtgcgcttcggggcccgcgccgggttgatgcaccccggaacgggctacagcgtcgcagcgtcgctcctgtgcgcagatgttctagcccgcgatcttcgtgacggttcgaacgcgcgactgtggtcgtggaaggcccgcaccgtcgcgtccctgcggtcggttggtctccgcgtcctcctgaatctggatgcggaacacgtccccgaattcttcgagcatttcttcgatctgccgccacaactccaggcggcatatctatctgaacgatcgaatccgcgcggcaccctgtcggcaatggggcggatgtttcgggcatcctccggcgatatccgcagtgtgattctccgttcgacgcttcgccgggaagttcgatga

SEQ ID NO: 5 - DNA, 1191 bp, Rhodococcus sp.
atgtttcgacagggatctcggcccccgtgtcaggcttgctgcgtgacagcatctccccgcccagcgatctgggacgttgcgatcgtgggcctcgggcctgccggccgcgccctggcacacagggccacggctctcggtgcgacggtcgtctcggtcgacccactcccccgcagatcgtggaccgcgacgtacgcggcgtgggaagacgagctccccagctggctcccgagggacgcggtcgaaacgagaacttctcgcccggcagcctggacgacacgtcgccacatcatcgatcgcacctattgtgttttgaatacacccttactgcaatcaatactcagtagggaatgtggaactgttctcccaggcagagccgtggacctctcccccacctccgtccagctggacggagggtccacgctggaggcgcatgtcgtcgtcgacgcccgcggcaccggcgacagccccgacctcgcccagcagacggcatacggcgtcgtggtggaccccgacacggccgcacatgcgctcgccggcgaggccgcggtgttcatggactggcgccgcgacaacggcacatcaccctcggacgcgccgtccttcctctacggggttccgctcgcgcacgaccggttcctgctcgaagagacctgcctggtcggtcgcccgccactcccgatctcggaactcgccacccgactccacgaccgactccgacagcggggcgtcaccgcgccgtcccattcggacgtcgagagagtcagattcgccgtcgagccacctcccgaggcgggtcgtgcccacagctcggatcaggtgcgcttcggggcccgcgccgggttgatgcaccccggaacgggctacagcgtcgcagcgtcgctcctgtgcgcagatgttctagcccgcgatcttcgtgacggttcgaacgcgcgactgtggtcgtggaaggcccgcaccgtcgcgtccctgcggtcggttggtctccgcgtcctcctgaatctggatgcggaacacgtccccgaattcttcgagcatttcttcgatctgccgccacaactccaggcggcatatctatctgaacgatcgaatccgcgcggcaccctgtcggcaatggggcggatgtttcgggcatcctccggcgatatccgcagtgtgattctccgttcgacgcttcgccgggaagttcgatga

SEQ ID NO: 6 - DNA, 1308 bp, Deinococcus sp. [sequence omitted]

SEQ ID NO: 7 - DNA, 1308 bp, Deinococcus sp. [sequence omitted]